(19) United States
(12) Patent Application Publication
(10) Pub. No.: US 2002/0169136 A1
(43) Pub. Date: Nov. 14, 2002

(54) COMPOUNDS AND METHODS FOR THE DIAGNOSIS AND TREATMENT OF B. MICROTI INFECTION

(76) Inventors: STEVEN G. REED, BELLEVUE, WA (US); MICHAEL J. LODES, SEATTLE, WA (US); RAYMOND L. HOUGHTON, BOTHELL, WA (US); PAUL R. SLEATH, SEATTLE, WA (US); PATRICIA D. MCNEIL, DES MOINES, WA (US)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 09/286,488

(22) Filed: Apr. 5, 1999

Related U.S. Application Data
(63) Continuation-in-part of application No. 08/990,571, filed on Dec. 11, 1997, now Pat. No. 6,214,971.
Application No. 08/990,571 is a continuation-in-part of application No. 08/845,258, filed on Apr. 24, 1997, now Pat. No. 6,183,976.
Application No. 08/845,258 is a continuation-in-part of application No. 08/723,142, filed on Oct. 1, 1996, now Pat. No. 6,306,396.

(51) Int. Cl. A61K 31/70; A01N 43/04

(52) U.S. Cl. 514/44

(57) ABSTRACT

Compounds and methods for the diagnosis and treatment of B. microti infection are disclosed. The compounds provided include polypeptides that contain at least one antigenic portion of a B. microti antigen and DNA sequences encoding such polypeptides. Antigenic epitopes of such antigens are also provided, together with pharmaceutical compositions and vaccines comprising such polypeptides, DNA sequences or antigenic epitopes. Diagnostic kits containing such polypeptides, DNA sequences or antigenic epitopes and a suitable detection reagent may be used for the detection of B. microti infection in patients and biological samples. Antibodies directed against such polypeptides and antigenic epitopes are also provided.

### CROSS-REFERENCE TO RELATED APPLICATIONS

This application is a continuation-in-part of U.S. application Ser. No. 08/990,571, filed Dec. 11, 1997, is a continuation-in-part of U.S. application Ser. No. 08/845,258, filed Apr. 24, 1997, which is a continuation-in-part of U.S. application Ser. No. 08/723,142 filed Oct. 1, 1996.

### TECHNICAL FIELD

The present invention relates generally to the detection of Babesia microti infection. In particular, the invention is related to polypeptides comprising a B. microti antigen, to antigenic epitopes of such an antigen and the use of such polypeptides and antigenic epitopes for the serodiagnosis and treatment of B. microti infection.

### BACKGROUND OF THE INVENTION

Babesiosis is a malaria-like illness caused by the rodent parasite Babesia microti (B. microti) which is generally transmitted to humans by the same tick that is responsible for the transmission of Lyme disease and ehrlichiosis, thereby leading to the possibility of co-infection with babesiosis, Lyme disease and ehrlichiosis from a single tick bite. While the number of reported cases of B. microti infection in the United States is increasing rapidly, infection with B. microti, including co-infection with Lyme disease, often remains undetected for extended periods of time. Babesiosis is potentially fatal, particularly in the elderly and in patients with suppressed immune systems. Patients infected with both Lyme disease and babesiosis have more severe symptoms and prolonged illness compared to those with either infection alone.

The preferred treatments for Lyme disease, ehrlichiosis and babesiosis are different, with penicillins, such as doxycycline and amoxicillin, being most effective in treating Lyme disease, tetracycline being preferred for the treatment of ehrlichiosis, and anti-malarial drugs, such as quinine and clindamycin, being most effective in the treatment of babesiosis. Accurate and early diagnosis of B. microti infection is thus critical but methods currently employed for diagnosis are problematic.

All three tick-borne illnesses share the same flu-like symptoms of muscle aches, fever, headaches and fatigue, thus making clinical diagnosis difficult. Microscopic analysis of blood samples may provide false-negative results when patients are first seen in the clinic. Indirect fluorescent antibody staining methods for total immunoglobulins to B. microti may be used to diagnose babesiosis infection, but such methods are time-consuming and expensive. There thus remains a need in the art for improved methods for the detection of B. microti infection.

### SUMMARY OF THE INVENTION

The present invention provides compositions and methods for the diagnosis and treatment of B. microti infection. In one aspect, polypeptides are provided comprising an immunogenic portion of a B. microti antigen, or a variant of such an antigen that differs only in conservative substitutions and/or modifications. In one embodiment, the antigen comprises an amino acid sequence encoded by a DNA sequence selected from the group consisting of (a) sequences recited in SEQ ID NO: 1-17, 37, 40, 42, 45, 50 and 51; (b) the complements of said sequences; and (c) sequences that hybridize to a sequence of (a) or (b) under moderately stringent conditions.

In another aspect, the present invention provides an antigenic epitope of a B. microti antigen comprising the amino acid sequence -X₁-X₂-X₃-X₄-X₅-Ser- (SEQ ID NO: 35), wherein X₁ is Glu or Gly, X₂ is Ala or Thr, X₃ is Gly or Val, X₄ is Trp or Gly and X₅ is Pro or Ser. In one embodiment of this aspect, X₁ is Glu, X₂ is Ala and X₃ is Gly. In a second embodiment X₁ is Gly, X₂ is Thr and X₅ is Pro. The present invention further provides polypeptides comprising at least two of the above antigenic epitopes, the epitopes being contiguous.

In yet another aspect, the present invention provides an antigenic epitope of a B. microti antigen comprising an amino acid sequence selected from the group consisting of SEQ ID NO: 36 and 39, together with polypeptides comprising at least two such antigenic epitopes, the epitopes being contiguous.

In a related aspect, DNA sequences encoding the above polypeptides, recombinant expression vectors comprising these DNA sequence and host cells transformed or transfected with such expression vectors are also provided.

In another aspect, the present invention provides fusion proteins comprising either a first and a second inventive polypeptide, a first and a second inventive antigenic epitope, or, alternatively, an inventive polypeptide and an inventive antigenic epitope.

In further aspects of the subject invention, methods and diagnostic kits are provided for detecting B. microti infection in a patient. In one embodiment, the method comprises: (a) contacting a biological sample with at least one polypeptide comprising an immunogenic portion of a B. microti antigen; and (b) detecting in the sample the presence of antibodies that bind to the polypeptide, thereby detecting B. microti infection in the biological sample. In other embodiments, the methods comprise: (a) contacting a biological sample with at least one of the above polypeptides or antigenic epitopes; and (b) detecting in the sample the presence of antibodies that bind to the polypeptide or antigenic epitope. Suitable biological samples include whole blood, sputum, serum, plasma, saliva, cerebrospinal fluid and urine. The diagnostic kits comprise one or more of the above polypeptides or antigenic epitopes in combination with a detection reagent.

The present invention also provides methods for detecting B. microti infection comprising: (a) obtaining a biological sample from a patient; (b) contacting the sample with at least two oligonucleotide primers in a polymerase chain reaction, at least one of the oligonucleotide primers being specific for a DNA sequence encoding the above polypeptides; and (c) detecting in the sample a DNA sequence that amplifies in the presence of the first and second oligonucleotide primers. In one embodiment, the oligonucleotide primer comprises at least about 10 contiguous nucleotides of a DNA sequence encoding the above polypeptides.

In a further aspect, the present invention provides a method for detecting B. microti infection in a patient comprising: (a) obtaining a biological sample from the patient; (b) contacting the sample with an oligonucleotide probe specific for a DNA sequence encoding the above polypeptides; and (c) detecting in the sample a DNA sequence that hybridizes to the oligonucleotide probe. In one embodiment of this aspect, the oligonucleotide probe comprises at least about 15 contiguous nucleotides of a DNA sequence encoding the above polypeptides.

In yet another aspect, the present invention provides antibodies, both polyclonal and monoclonal, that bind to the polypeptides described above, as well as methods for their use in the detection of B. microti infection.

Within other aspects, the present invention provides pharmaceutical compositions that comprise one or more of the above polypeptides or antigenic epitopes, or a DNA molecule encoding such polypeptides, and a physiologically acceptable carrier. The invention also provides vaccines comprising one or more of the inventive polypeptides or antigenic epitopes and a non-specific immune response enhancer, together with vaccines comprising one or more DNA sequences encoding such polypeptides and a non-specific immune response enhancer.

In yet another aspect, methods are provided for inducing protective immunity in a patient, comprising administering to a patient an effective amount of one or more of the above pharmaceutical compositions or vaccines.

These and other aspects of the present invention will become apparent upon reference to the following detailed description and attached drawings. All references disclosed herein are hereby incorporated by reference in their entirety as if each was incorporated individually.

### BRIEF DESCRIPTION OF THE DRAWINGS

FIG. 1 shows the genomic sequence of the B. microti antigen BMNI-3 (SEQ ID NO: 3) including a translation of the putative open reading frame (SEQ ID NO: 49). An internal six amino acid repeat sequence (SEQ ID NO: 35) is indicated by vertical lines within the open reading frame.

FIG. 2a shows the reactivity of the B. microti antigens BMNI-3 and BMNI-6, and the peptides BABS-1 and BABS-4 with sera from B. microti-infected individuals and from normal donors as determined by ELISA.

FIG. 2b shows the reactivity of the B. microti antigens BMNI-4 and BMNI-15 with sera from B. microti-infected individuals and from normal donors as determined by ELISA.

FIG. 3 shows the reactivity of the B. microti antigens MN-10 and BMNI-20 with sera from B. microti-infected patients and from normal donors as determined by ELISA.

FIG. 4 shows the results of Western blot analysis of representative B. microti antigens of the present invention.

FIG. 5 shows the reactivity of purified recombinant B. microti antigen BMNI-3 with sera from B. microti-infected patients, Lyme disease-infected patients, ehrlichiosis-infected patients and normal donors as determined by Western blot analysis.

FIG. 6 shows an alignment of the repeat region of different homologues of the B. microti antigen BMNI-6, illustrating the geographic variation in the number and location of the repeats.

### DETAILED DESCRIPTION OF THE INVENTION

As noted above, the present invention is generally directed to compositions and methods for the diagnosis and treatment of B. microti infection. In one aspect, the compositions of the subject invention include polypeptides that comprise at least one immunogenic portion of a B. microti antigen, or a variant of such an antigen that differs only in conservative substitutions and/or modifications.

As used herein, the term “polypeptide” encompasses amino acid chains of any length, including full length proteins (i.e., antigens), wherein the amino acid residues are linked by covalent peptide bonds. Thus, a polypeptide comprising an immunogenic portion of one of the above antigens may consist entirely of the immunogenic portion, or may contain additional sequences. The additional sequences may be derived from the native B. microti antigen or may be heterologous, and such sequences may (but need not) be immunogenic.

An “immunogenic portion” of an antigen is a portion that is capable of reacting with sera obtained from a B. microti-infected individual (i.e., generates an absorbance reading with sera from infected individuals that is at least three standard deviations above the absorbance obtained with sera from uninfected individuals, in a representative ELISA assay described herein). Polypeptides comprising at least an immunogenic portion of one or more B. microti antigens as described herein may generally be used, alone or in combination, to detect B. microti in a patient.

The compositions and methods of the present invention also encompass variants of the above polypeptides and DNA molecules. Such variants include, but are not limited to, naturally occurring allelic variants of the inventive sequences.

A polypeptide “variant,” as used herein, is a polypeptide that differs from the recited polypeptide only in conservative substitutions and/or modifications, such that the antigenic properties of the polypeptide are retained. In a preferred embodiment, variant polypeptides differ from an identified sequence by substitution, deletion or addition of five amino acids or fewer. Such variants may generally be identified by modifying one of the above polypeptide sequences, and evaluating the antigenic properties of the modified polypeptide using, for example, the representative procedures described herein. Polypeptide variants preferably exhibit at least about 70%, more preferably at least about 90% and most preferably at least about 95% identity (determined as described below) to the identified polypeptides.

As used herein, a “conservative substitution” is one in which an amino acid is substituted for another amino acid that has similar properties, such that one skilled in the art of peptide chemistry would expect the secondary structure and hydropathic nature of the polypeptide to be substantially unchanged. In general, the following groups of amino acids represent conservative changes: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his.

Variants may also, or alternatively, contain other modifications, including the deletion or addition of amino acids that have minimal influence on the antigenic properties, secondary structure and hydropathic nature of the polypeptide. For example, a polypeptide may be conjugated to a signal (or leader) sequence at the N-terminal end of the protein which co-translationally or post-translationally directs transfer of the protein. The polypeptide may also be conjugated to a linker or other sequence for ease of synthesis, purification or identification of the polypeptide (e.g., poly-His), or to enhance binding of the polypeptide to a solid support. For example, a polypeptide may be conjugated to an immunoglobulin Fc region.

A nucleotide “variant” is a sequence that differs from the recited nucleotide sequence in having one or more nucleotide deletions, substitutions or additions. Such modifications may be readily introduced using standard mutagenesis techniques, such as oligonucleotide-directed site-specific mutagenesis as taught, for example, by Adelman et al. (DNA, 2:183, 1983). Nucleotide variants may be naturally occurring allelic variants, or non-naturally occurring variants. Variant nucleotide sequences preferably exhibit at least about 70%, more preferably at least about 80% and most preferably at least about 90% identity (determined as described below) to the recited sequence.

Two nucleotide or polypeptide sequences are said to be “identical” if the sequence of nucleotides or amino acid residues in the two sequences is the same when aligned for maximum correspondence as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Resarch Foundaiton, Washington D.C. Vol. 5, Suppl. 3, pp. 345-358; Hein J. (1990) Unified Approach to Alignment and Phylogenes pp. 626-645 Methods in Enzymology vol. 183, Academic Press, Inc., San Diego, Calif.; Higgins, D. G. and Sharp, P. M. (1989) Fast and sensitive multiple sequence alignments on a microcomputer CABIOS 5:151-153; Myers, E. W. and Muller W. (1988) Optimal alignments in linear space CABIOS 4:11-17; Robinson, E. D. (1971) Comb. Theor 11:105; Santou, N. Nes, M. (1987) The neighbor joining method. A new method for reconstructing phylogenetic trees Mol. Biol. Evol. 4:406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Rapid similarity searches of nucleic acid and protein data banks Proc. Natl. Acad., Sci. USA 80:726-730.

Preferably, the “percentage of sequence identity” is determined by comparing two optimally aligned sequences over a window of comparison of at least 20 positions, wherein the portion of the polynucleotide sequence in the comparison window may comprise additions or deletions (i.e. gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (i.e. the window size) and multiplying the results by 100 to yield the percentage of sequence identity.

In specific embodiments, the subject invention discloses polypeptides comprising at least an immunogenic portion of a B. microti antigen (or a variant of such an antigen), that comprises one or more of the amino acid sequences encoded by (a) a DNA sequence selected from the group consisting of SEQ ID NO: 1-17, 37, 40, 42, 45 50, 51 and 56-67, (b) the complements of such DNA sequences or (c) DNA sequences substantially homologous to a sequence in (a) or (b).

The B. microti antigens provided by the present invention include variants that are encoded by DNA sequences which are substantially homologous to one or more of the DNA sequences specifically recited herein. “Substantial homology,” as used herein, refers to DNA sequences that are capable of hybridizing under moderately stringent conditions. Suitable moderately stringent conditions include prewashing in a solution of 5× SSC, 0.5% SDS. 1.0 mM EDTA (pH 8.0); hybridizing at 50° C.-65° C., 5× SSC, overnight or, in the event of cross-species homology, at 45° C. with 0.5× SSC; followed by washing twice at 65° C. for 20 minutes with each of 2×, 0.5× and 0.2× SSC containing 0.1% SDS. Such hybridizing DNA sequences are also within the scope of this invention, as are nucleotide sequences that, due to code degeneracy, encode an immunogenic polypeptide that is encoded by a hybridizing DNA sequence.

In general, B. microti antigens, and DNA sequences encoding such antigens, may be prepared using any of a variety of procedures. For example, DNA molecules encoding B. microti antigens may be isolated from a B. microti genomic or cDNA expression library by screening with sera from B. microti-infected individuals as described below in Example 1, and sequenced using techniques well known to those of skill in the art. DNA molecules encoding B. microti antigens may also be isolated by screening an appropriate B. microti expression library with anti-sera (e.g., rabbit) raised specifically against B. microti antigens.

Antigens may be induced from such clones and evaluated for a desired property, such as the ability to react with sera obtained from a B. microti-infected individual as described herein. Alternatively, antigens may be produced recombinantly, as described below, by inserting a DNA sequence that encodes the antigen into an expression vector and expressing the antigen in an appropriate host. Antigens may be partially sequenced using, for example, traditional Edman chemistry. See Edman and Berg, Eur. J. Biochem. 80:116-132, 1967.

DNA sequences encoding antigens may also be obtained by screening an appropriate B. microti cDNA or genomic DNA library for DNA sequences that hybridize to degenerate oligonucleotides derived from partial amino acid sequences of isolated antigens. Degenerate oligonucleotide sequences for use in such a screen may be designed and synthesized, and the screen may be performed, as described (for example) in Sambrook et al., Molecular Cloning: A Laboratory Manual, Cold Spring Harbor Laboratories, Cold Spring Harbor, N.Y. (and references cited therein). Polymerase chain reaction (PCR) may also be employed, using the above oligonucleotides in methods well known in the art, to isolate a nucleic acid probe from a cDNA or genomic library. The library screen may then be performed using the isolated probe.

Synthetic polypeptides having fewer than about 100 amino acids, and generally fewer than about 50 amino acids, may be generated using techniques well known in the art. For example, such polypeptides may be synthesized using any of the commercially available solid-phase techniques, such as the Merrifield solid-phase synthesis method, where amino acids are sequentially added to a growing amino acid chain. See Merrifield, J. Am. Chem. Soc. 85:2149-2146, 1963. Equipment for automated synthesis of polypeptides is commercially available from suppliers such as Applied BioSystems, Inc., Foster City, Calif., and may be operated according to the manufacturer's instructions.

Immunogenic portions of B. microti antigens may be prepared and identified using well known techniques, such as those summarized in Paul, Fundamental Immunology, 3d ed., Raven Press, 1993, pp. 243-247 and references cited therein. Such techniques include screening polypeptide portions of the native antigen for immunogenic properties. The representative ELISAs described herein may generally be employed in these screens. An immunogenic portion of a polypeptide is a portion that, within such representative assays, generates a signal in such assays that is substantially similar to that generated by the full length antigen. In other words, an immunogenic portion of a B. microti antigen generates at least about 20%, and preferably about 100%, of the signal induced by the full length antigen in a model ELISA as described herein.

Portions and other variants of B. microti antigens may be generated by synthetic or recombinant means. Variants of a native antigen may generally be prepared using standard mutagenesis techniques, such as oligonucleotide-directed site-specific mutagenesis. Sections of the DNA sequence may also be removed using standard techniques to permit preparation of truncated polypeptides.

Recombinant polypeptides containing portions and/or variants of a native antigen may be readily prepared from a DNA sequence encoding the polypeptide using a variety of techniques well known to those of ordinary skill in the art. For example, supernatants from suitable host/vector systems which secrete recombinant protein into culture media may be first concentrated using a commercially available filter. Following concentration, the concentrate may be applied to a suitable purification matrix such as an affinity matrix or an ion exchange resin. Finally, one or more reverse phase HPLC steps can be employed to further purify a recombinant protein.

Any of a variety of expression vectors known to those of ordinary skill in the art may be employed to express recombinant polypeptides as described herein. Expression may be achieved in any appropriate host cell that has been transformed or transfected with an expression vector containing a DNA molecule that encodes a recombinant polypeptide. Suitable host cells include prokaryotes, yeast and higher eukaryotic cells. Preferably, the host cells employed are E. coli; yeast or a mammalian cell line, such as COS or CHO. The DNA sequences expressed in this manner may encode naturally occurring antigens, portions of naturally occurring antigens, or other variants thereof.

In another aspect, the present invention provides epitope repeat sequences, or antigenic epitopes, of a B. microti antigen, together with polypeptides comprising at least two such contiguous antigenic epitopes. As used herein an “epitope” is a portion of an antigen that reacts with sera from B. microti-infected individuals (i.e. an epitope is specifically bound by one or more antibodies present in such sera). As discussed above, epitopes of the antigens described in the present application may be generally identified using techniques well known to those of skill in the art.

In one embodiment, antigenic epitopes of the present invention comprise the amino acid sequence -X₁-X₂-X₃-X₄-X₅-Ser-(SEQ ID NO: 35), wherein X₁ is Glu or Gly, X₂ is Ala or Thr, X₃ is Gly or Val, X₄ is Trp or Gly, and X₅ is Pro or Ser. In another embodiment, the antigenic epitopes of the present invention comprise an amino acid sequence selected from the group consisting of SEQ ID NO: 36 and 39. As discussed in more detail below, antigenic epitopes provided herein may be employed in the diagnosis and treatment of B. microti infection, either alone or in combination with other B. microti antigens or antigenic epitopes. Antigenic epitopes and polypeptides comprising such epitopes may be prepared by synthetic means, as described generally above and in detail in Example 2.

In general, regardless of the method of preparation, the polypeptides and antigenic epitopes disclosed herein are prepared in substantially pure form. Preferably, the polypeptides and antigenic epitopes are at least about 80% pure, more preferably at least about 90% pure and most preferably at least about 99% pure.

In a further aspect, the present invention provides fusion proteins comprising either a first and a second inventive polypeptide, a first and a second inventive antigenic epitope or an inventive polypeptide and an antigenic epitope of the present invention, together with variants of such fusion proteins. The fusion proteins of the present invention may also include a linker peptide between the polypeptides or antigenic epitopes.

A DNA sequence encoding a fusion protein of the present invention is constructed using known recombinant DNA techniques to assemble separate DNA sequences encoding, for example, the first and second polypeptides into an appropriate expression vector. The 3′ end of a DNA sequence encoding the first polypeptide is ligated, with or without a peptide linker, to the 5′ end of a DNA sequence encoding the second polypeptide so that the reading frames of the sequences are in phase to permit mRNA translation of the two DNA sequences into a single fusion protein that retains the biological activity of both the first and the second polypeptides.

A peptide linker sequence may be employed to separate the first and the second polypeptides by a distance sufficient to ensure that each polypeptide folds into its secondary and tertiary structures. Such a peptide linker sequence is incorporated into the fusion protein using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39-46, 1985; Murphy et al., Proc. Natl. Acad. Sci. USA 83:8258-8562, 1986; U.S. Pat. No. 4,935,233 and U.S. Pat. No. 4,751,180. The linker sequence may be from 1 to about 50 amino acids in length. Peptide linker sequences are not required when the first and second polypeptides have non-essential N-terminal amino acid regions that can be used to separate the functional domains and prevent steric hindrance.

In another aspect, the present invention provides methods for using polypeptides comprising an immunogenic portion of a B. microti antigen and the antigenic epitopes described above to diagnose babesiosis. In this aspect, methods are provided for detecting B. microti infection in a biological sample, using one or more of the above polypeptides and antigenic epitopes, alone or in combination. For clarity, the term “polypeptide” will be used when describing specific embodiments of the inventive diagnostic methods. However, it will be clear to one of skill in the art that the antigenic epitopes of the present invention may also be employed in such methods.

As used herein, a “biological sample” is any antibody-containing sample obtained from a patient. Preferably, the sample is whole blood, sputum, serum, plasma, saliva, cerebrospinal fluid or urine. More preferably, the sample is a blood, serum or plasma sample obtained from a patient. The polypeptides are used in an assay, as described below, to determine the presence or absence of antibodies to the polypeptide(s) in the sample, relative to a predetermined cut-off value. The presence of such antibodies indicates previous sensitization to B. microti antigens which may be indicative of babesiosis.

In embodiments in which more than one polypeptide is employed, the polypeptides used are preferably complementary (i.e., one component polypeptide will tend to detect infection in samples where the infection would not be detected by another component polypeptide). Complementary polypeptides may generally be identified by using each polypeptide individually to evaluate serum samples obtained from a series of patients known to be infected with B. microti. After determining which samples test positive (as described below) with each polypeptide, combinations of two or more polypeptides may be formulated that are capable of detecting infection in most, or all, of the samples tested.

A variety of assay formats are known to those of ordinary skill in the art for using one or more polypeptides to detect antibodies in a sample. See, e.g., Harlow and Lane, Antibodies: A Laboratory Manual, Cold Spring Harbor Laboratory, 1988, which is incorporated herein by reference. In a preferred embodiment, the assay involves the use of polypeptide immobilized on a solid support to bind to and remove the antibody from the sample. The bound antibody may then be detected using a detection reagent that contains a reporter group. Suitable detection reagents include antibodies that bind to the antibody/polypeptide complex and free polypeptide labeled with a reporter group (e.g., in a semi-competitive assay). Alternatively, a competitive assay may be utilized, in which an antibody that binds to the polypeptide is labeled with a reporter group and allowed to bind to the immobilized antigen after incubation of the antigen with the sample. The extent to which components of the sample inhibit the binding of the labeled antibody to the polypeptide is indicative of the reactivity of the sample with the immobilized polypeptide.

The solid support may be any solid material known to those of ordinary skill in the art to which the antigen may be attached. For example, the solid support may be a test well in a microtiter plate, or a nitrocellulose or other suitable membrane. Alternatively, the support may be a bead or disc, such as glass, fiberglass, latex or a plastic material such as polystyrene or polyvinylchloride. The support may also be a magnetic particle or a fiber optic sensor, such as those disclosed, for example, in U.S. Pat. No. 5,359,681.

The polypeptides may be bound to the solid support using a variety of techniques known to those of ordinary skill in the art. In the context of the present invention, the term “bound” refers to both noncovalent association, such as adsorption, and covalent attachment (which may be a direct linkage between the antigen and functional groups on the support or may be a linkage by way of a cross-linking agent). Binding by adsorption to a well in a microtiter plate or to a membrane is preferred. In such cases, adsorption may be achieved by contacting the polypeptide, in a suitable buffer, with the solid support for a suitable amount of time. The contact time varies with temperature, but is typically between about 1 hour and 1 day. In general, contacting a well of a plastic microtiter plate (such as polystyrene or polyvinylchloride) with an amount of polypeptide ranging from about 10 ng to about 1 μg, and preferably about 100 ng, is sufficient to bind an adequate amount of antigen.

Covalent attachment of polypeptide to a solid support may generally be achieved by first reacting the support with a bifunctional reagent that will react with both the support and a functional group, such as a hydroxyl or amino group, on the polypeptide. For example, the polypeptide may be bound to supports having an appropriate polymer coating using benzoquinone or by condensation of an aldehyde group on the support with an amine and an active hydrogen on the polypeptide (see, e.g., Pierce Immunotechnology Catalog and Handbook, 1991, at A12-A13).

In certain embodiments, the assay is an enzyme linked immunosorbent assay (ELISA). This assay may be performed by first contacting a polypeptide antigen that has been immobilized on a solid support, commonly the well of a microtiter plate, with the sample, such that antibodies to the polypeptide within the sample are allowed to bind to the immobilized polypeptide. Unbound sample is then removed from the immobilized polypeptide and a detection reagent capable of binding to the immobilized antibody-polypeptide complex is added. The amount of detection reagent that remains bound to the solid support is then determined using a method appropriate for the specific detection reagent.

More specifically, once the polypeptide is immobilized on the support as described above, the remaining protein binding sites on the support are typically blocked. Any suitable blocking agent known to those of ordinary skill in the art, such as bovine serum albumin (BSA) or Tween 20™ (Sigma Chemical Co., St. Louis, Mo.) may be employed. The immobilized polypeptide is then incubated with the sample, and antibody is allowed to bind to the antigen. The sample may be diluted with a suitable diluent, such as phosphate-buffered saline (PBS) prior to incubation. In general, an appropriate contact time (i.e., incubation time) is that period of time that is sufficient to detect the presence of antibody within a B. microti-infected sample. Preferably, the contact time is sufficient to achieve a level of binding that is at least 95% of that achieved at equilibrium between bound and unbound antibody. Those of ordinary skill in the art will recognize that the time necessary to achieve equilibrium may be readily determined by assaying the level of binding that occurs over a period of time. At room temperature, an incubation time of about 30 minutes is generally sufficient.

Unbound sample may then be removed by washing the solid support with an appropriate buffer, such as PBS containing 0.1% Tween 20™. Detection reagent may then be added to the solid support. An appropriate detection reagent is any compound that binds to the immobilized antibody-polypeptide complex and that can be detected by any of a variety of means known to those in the art. Preferably, the detection reagent contains a binding agent (such as, for example, Protein A, Protein G, immunoglobulin, lectin or free antigen) conjugated to a reporter group. Preferred reporter groups include enzymes (such as horseradish peroxidase), substrates, cofactors, inhibitors, dyes, radionuclides, luminescent groups, fluorescent groups and biotin. The conjugation of binding agent to reporter group may be achieved using standard methods known to those of ordinary skill in the art. Common binding agents may also be purchased conjugated to a variety of reporter groups from many commercial sources (e.g., Zymed Laboratories, San Francisco, Calif., and Pierce, Rockford, Ill.).

The detection reagent is then incubated with the immobilized antibody-polypeptide complex for an amount of time sufficient to detect the bound antibody. An appropriate amount of time may generally be determined from the manufacturer's instructions or by assaying the level of binding that occurs over a period of time. Unbound detection reagent is then removed and bound detection reagent is detected using the reporter group. The method employed for detecting the reporter group depends upon the nature of the reporter group. For radioactive groups, scintillation counting or autoradiographic methods are generally appropriate. Spectroscopic methods may be used to detect dyes. luminescent groups and fluorescent groups. Biotin may be detected using avidin, coupled to a different reporter group (commonly a radioactive or fluorescent group or an enzyme). Enzyme reporter groups may generally be detected by the addition of substrate (generally for a specific period of time), followed by spectroscopic or other analysis of the reaction products.

To determine the presence or absence of anti-B. microti antibodies in the sample, the signal detected from the reporter group that remains bound to the solid support is generally compared to a signal that corresponds to a predetermined cut-off value. In one preferred embodiment, the cut-off value is the average mean signal obtained when the immobilized antigen is incubated with samples from an uninfected patient. In general, a sample generating a signal that is three standard deviations above the predetermined cut-off value is considered positive for babesiosis. In an alternate preferred embodiment, the cut-off value is determined using a Receiver Operator Curve, according to the method of Sackett et al., Clinical Epidemiology: A Basic Science for Clinical Medicine, Little Brown and Co., 1985, pp. 106-107. Briefly, in this embodiment, the cut-off value may be determined from a plot of pairs of true positive rates (i.e., sensitivity) and false positive rates (100%-specificity) that correspond to each possible cut-off value for the diagnostic test result. The cut-off value on the plot that is the closest to the upper left-hand corner (i.e., the value that encloses the largest area) is the most accurate cut-off value, and a sample generating a signal that is higher than the cut-off value determined by this method may be considered positive. Alternatively, the cut-off value may be shifted to the left along the plot, to minimize the false positive rate, or to the right, to minimize the false negative rate. In general, a sample generating a signal that is higher than the cut-off value determined by this method is considered positive for babesiosis.

In a related embodiment, the assay is performed in a rapid flow-through or strip test format, wherein the antigen is immobilized on a membrane, such as nitrocellulose. In the flow-through test, antibodies within the sample bind to the immobilized polypeptide as the sample passes through the membrane. A detection reagent (e.g., protein A-colloidal gold) then binds to the antibody-polypeptide complex as the solution containing the detection reagent flows through the membrane. The detection of bound detection reagent may then be performed as described above. In the strip test format, one end of the membrane to which polypeptide is bound is immersed in a solution containing the sample. The sample migrates along the membrane through a region containing detection reagent and to the area of immobilized polypeptide. Concentration of detection reagent at the polypeptide indicates the presence of anti-B. microti antibodies in the sample. Typically, the concentration of detection reagent at that site generates a pattern, such as a line, that can be read visually. The absence of such a pattern indicates a negative result. In general, the amount of polypeptide immobilized on the membrane is selected to generate a visually discernible pattern when-the biological sample contains a level of antibodies that would be sufficient to generate a positive signal in an ELISA, as discussed above. Preferably, the amount of polypeptide immobilized on the membrane ranges from about 25 ng to about 1 μg, and more preferably from about 50 ng to about 500 ng. Such tests can typically be performed with a very small amount (e.g., one drop) of patient serum or blood.

Of course, numerous other assay protocols exist that are suitable for use with the polypeptides and antigenic epitopes of the present invention. The above descriptions are intended to be exemplary only.

In yet another aspect, the present invention provides antibodies to the polypeptides and antigenic epitopes of the present invention. Antibodies may be prepared by any of a variety of techniques known to those of ordinary skill in the art. See, e.g., Harlow and Lane, Antibodies: A Laboratory Manual, Cold Spring Harbor Laboratory, Cold Spring Harbor, N.Y., 1988. In one such technique, an immunogen comprising the antigenic polypeptide or epitope is initially injected into any of a wide variety of mammals (e.g., mice, rats, rabbits, sheep and goats). The polypeptides and antigenic epitopes of this invention may serve as the immunogen without modification. Alternatively, particularly for relatively short polypeptides, a superior immune response may be elicited if the polypeptide is joined to a carrier protein, such as bovine serum albumin or keyhole limpet hemocyanin. The immunogen is injected into the animal host, preferably according to a predetermined schedule incorporating one or more booster immunizations, and the animals are bled periodically. Polyclonal antibodies specific for the polypeptide or antigenic epitope may then be purified from such antisera by, for example, affinity chromatography using the polypeptide or antigenic epitope coupled to a suitable solid support.

Monoclonal antibodies specific for the antigenic polypeptide or epitope of interest may be prepared, for example, using the technique of Kohler and Milstein, Eur. J. Immunol. 6:511-519, 1976, and improvements thereto. Briefly, these methods involve the preparation of immortal cell lines capable of producing antibodies having the desired specificity (i.e., reactivity with the polypeptide or antigenic epitope of interest). Such cell lines may be produced, for example, from spleen cells obtained from an animal immunized as described above. The spleen cells are then immortalized by, for example, fusion with a myeloma cell fusion partner, preferably one that is syngeneic with the immunized animal. A variety of fusion techniques may be employed. For example, the spleen cells and myeloma cells may be combined with a nonionic detergent for a few minutes and then plated at low density on a selective medium that supports the growth of hybrid cells, but not myeloma cells. A preferred selection technique uses HAT (hypoxanthine, aminopterin, thymidine) selection. After a sufficient time, usually about 1 to 2 weeks, colonies of hybrids are observed. Single colonies are selected and tested for binding activity against the polypeptide or antigenic epitope. Hybridomas having high reactivity and specificity are preferred.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction. The polypeptides or antigenic epitopes of this invention may be used in the purification process in, for example, an affinity chromatography step.

Antibodies may be used in diagnostic tests to detect the presence of B. microti antigens using assays similar to those detailed above and other techniques well known to those of skill in the art, thereby providing a method for detecting B. microti infection in a patient.

Diagnostic reagents of the present invention may also comprise DNA sequences encoding one or more of the above polypeptides, or one or more portions thereof. For example, at least two oligonucleotide primers may be employed in a polymerase chain reaction (PCR) based assay to amplify B. microti-specific cDNA derived from a biological sample, wherein at least one of the oligonucleotide primers is specific for a DNA molecule encoding a polypeptide of the present invention. The presence of the amplified cDNA is then detected using techniques well known in the art, such as gel electrophoresis. Similarly, oligonucleotide probes specific for a DNA molecule encoding a polypeptide of the present invention may be used in a hybridization assay to detect the presence of an inventive polypeptide in a biological sample.

As used herein, the term “oligonucleotide primer/probe specific for a DNA molecule” means an oligonucleotide sequence that has at least about 80%, preferably at least about 90% and more preferably at least about 95%, identity to the DNA molecule in question. Oligonucleotide primers and/or probes which may be usefully employed in the inventive diagnostic methods preferably have at least about 10-40 nucleotides. In a preferred embodiment, the oligonucleotide primers comprise at least about 10 contiguous nucleotides of a DNA molecule encoding one of the polypeptides disclosed herein. Preferably, oligonucleotide probes for use in the inventive diagnostic methods comprise at least about 15 contiguous oligonucleotides of a DNA molecule encoding one of the polypeptides disclosed herein. Techniques for both PCR based assays and hybridization assays are well known in the art (see, for example, Mullis et al. Ibid; Ehrlich, Ibid). Primers or probes may thus be used to detect B. microti-specific sequences in biological samples, preferably sputum, blood, serum, saliva, cerebrospinal fluid or urine. DNA probes or primers comprising oligonucleotide sequences described above may be used alone or in combination with each other.

In another aspect, the present invention provides methods for using one or more of the above polypeptides, antigenic epitopes or fusion proteins (or DNA molecules encoding such polypeptides) to induce protective immunity against B. microti infection in a patient. As used herein, a “patient” refers to any warm-blooded animal, preferably a human. A patient may be afflicted with a disease, or may be free of detectable disease and/or infection. In other words, protective immunity may be induced to prevent or treat babesiosis.

In this aspect, the polypeptide, antigenic epitope, fusion protein or DNA molecule is generally present within a pharmaceutical composition or a vaccine. Pharmaceutical compositions may comprise one or more polypeptides, each of which may contain one or more of the above sequences (or variants thereof), and a physiologically acceptable carrier. Vaccines may comprise one or more of the above polypeptides and a non-specific immune response enhancer, such as an adjuvant or a liposome (into which the polypeptide is incorporated). Such pharmaceutical compositions and vaccines may also contain other B. microti antigens, either incorporated into a combination polypeptide or present within a separate polypeptide.

Alternatively, a vaccine may contain DNA encoding one or more polypeptides, antigenic epitopes or fusion proteins as described above, such that the polypeptide is generated in situ. In such vaccines, the DNA may be present within any of a variety of delivery systems known to those of ordinary skill in the art, including nucleic acid expression systems, bacterial and viral expression systems. Appropriate nucleic acid expression systems contain the necessary DNA sequences for expression in the patient (such as a suitable promoter and terminating signal). Bacterial delivery systems involve the administration of a bacterium (such as Bacillus-Calmette-Guerrin) that expresses an immunogenic portion of the polypeptide on its cell surface. In a preferred embodiment, the DNA may be introduced using a viral expression system (e.g., vaccinia or other pox virus, retrovirus, or adenovirus), which may involve the use of a non-pathogenic (defective), replication competent virus. Techniques for incorporating DNA into such expression systems are well known to those of ordinary skill in the art. The DNA may also be “naked,” as described, for example, in Ulmer et al., Science 259:1745-1749, 1993 and reviewed by Cohen, Science 259:1691-1692, 1993. The uptake of naked DNA may be increased by coating the DNA onto biodegradable beads, which are efficiently transported into the cells.

In a related aspect, a DNA vaccine as described above may be administered simultaneously with or sequentially to either a polypeptide of the present invention or a known B. microti antigen. For example, administration of DNA encoding a polypeptide of the present invention, either “naked” or in a delivery system as described above, may be followed by administration of an antigen in order to enhance the protective immune effect of the vaccine.

Routes and frequency of administration, as well as dosage, will vary from individual to individual. In general, the pharmaceutical compositions and vaccines may be administered by injection (e.g., intracutaneous, intramuscular, intravenous or subcutaneous), intranasally (e.g., by aspiration) or orally. Between 1 and 3 doses may be administered for a 1-36 week period. Preferably, 3 doses are administered, at intervals of 3-4 months, and booster vaccinations may be given periodically thereafter. Alternate protocols may be appropriate for individual patients. A suitable dose is an amount of polypeptide or DNA that, when administered as described above, is capable of raising an immune response in an immunized patient sufficient to protect the patient from B. microti infection for at least 1-2 years. In general, the amount of polypeptide present in a dose (or produced in situ by the DNA in a dose) ranges from about 1 pg to about 100 mg per kg of host, typically from about 10 pg to about 1 mg, and preferably from about 100 pg to about 1 μg. Suitable dose sizes will vary with the size of the patient, but will typically range from about 0.1 mL to about 5 mL.

While any suitable carrier known to those of ordinary skill in the art may be employed in the pharmaceutical compositions of this invention, the type of carrier will vary depending on the mode of administration. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactic galactide) may also be employed as carriers for the pharmaceutical compositions of this invention. Suitable biodegradable microspheres are disclosed, for example, in U.S. Pat. Nos. 4,897,268 and 5,075,109.

Any of a variety of adjuvants may be employed in the vaccines of this invention to nonspecifically enhance the immune response. Most adjuvants contain a substance designed to protect the antigen from rapid catabolism, such as aluminum hydroxide or mineral oil, and a nonspecific stimulator of immune responses, such as lipid A, Bortadella pertussis or Mycobacterium tuberculosis. Suitable adjuvants are commercially available as, for example, Freund's Incomplete Adjuvant and Freund's Complete Adjuvant (Difco Laboratories, Detroit, Mich.) and Merck Adjuvant 65 (Merck and Company, Inc., Rahway, N.J.). Other suitable adjuvants include alum, biodegradable microspheres, monophosphoryl lipid A and quil A.

The following Examples are offered by way of illustration and not by way of limitation.

### EXAMPLE 1

### Isolation of DNA Sequences Encoding B. microti Antigens

This example illustrates the preparation of DNA sequences encoding B. microti antigens by screening a B. microti expression library with sera obtained from patients infected with B. microti.

B. microti genomic DNA was isolated from infected hamsters and sheared by sonication. The resulting randomly sheared DNA was used to construct a B. microti genomic expression library (approximately 0.5-4.0 kbp inserts) with EcoRI adaptors and a Lambda ZAP II/EcoRI/CIAP vector (Stratagene, La Jolla, Calif.). The unamplified library (1.2×10⁶/ml) was screened with an E. coli lysate-absorbed B. microti patient serum pool, as described in Sambrook et al., Molecular Cloning: A Laboratory Manual, Cold Spring Harbor Laboratories, Cold Spring Harbor, N.Y., 1989. Positive plaques were visualized and purified with goat-anti-human alkaline phosphatase. Phagemid from the plaques was rescued and DNA sequence for positive clones was obtained using forward, reverse, and specific internal primers on a Perkin Elmer/Applied Biosystems Inc. Automated Sequencer Model 373A (Foster City, Calif.).

Seventeen antigens (hereinafter referred to as BMNI-1-BMNI-17) were purified and three were possibly redundant. The determined DNA sequences for BMNI-1-BMNI-17 are shown in SEQ ID NO: 1-17, respectively. The deduced amino acid sequences for BMNI-1-BMNI-6, BMNI-8 and BMNI-10-BMNI-17 are shown in SEQ ID NO: 18-32, respectively, with the predicted 5′ and 3′ protein sequences for BMNI-9 being shown in SEQ ID NO: 33 and 34, respectively.

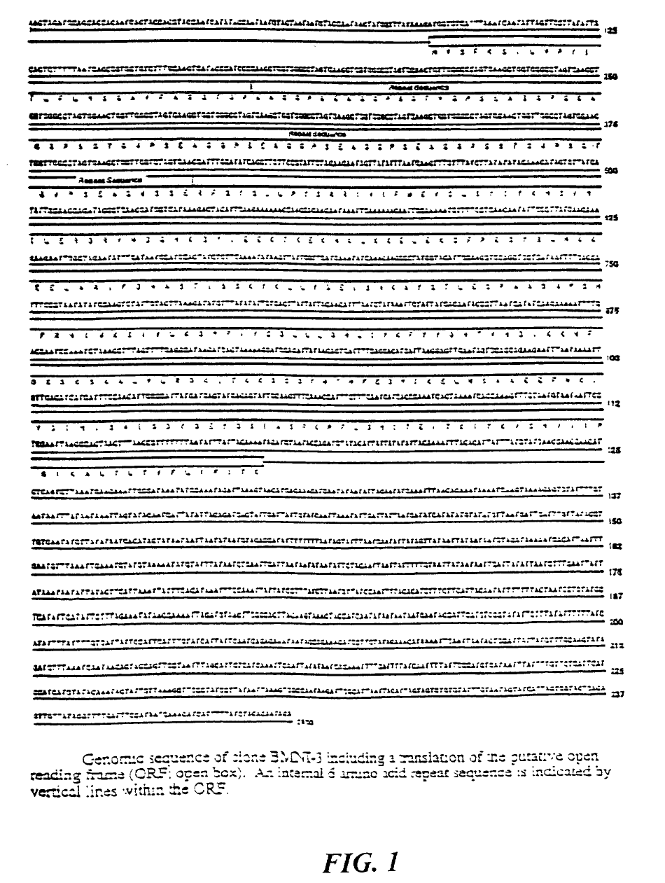

The isolated DNA sequences were compared to known sequences in the gene bank using the DNA STAR system. Nine of the seventeen antigens (BMNI-1, BMNI-2, BMNI-3, BMNI-5, BMNI-6, BMNI-7, BMNI-12, BMNI-13 and BMNI-16) share some homology, with BMNI-1 and BMNI-16 being partial clones of BMNI-3. All of these nine antigens contain a degenerate repeat of six amino acids (SEQ ID NO: 35), with between nine to twenty-two repeats occurring in each antigen. The repeat portion of the sequences was found to bear some similarity to a Plasmodium falciparum merozoite surface antigen (MSA-2 gene). FIG. 1 shows the genomic sequence of BMNI-3 including a translation of the putative open reading frame, with the internal six amino acid repeat sequence being indicated by vertical lines within the open reading frame.

A second group of five antigens bear some homology to each other but do not show homology to any previously identified sequences (BMNI-4, BMNI-8, BMNI-9, BMNI-10 and BMNI-11). These antigens may belong to a family of genes or may represent parts of a repetitive sequence. BMNI-17 contains a novel degenerate repeat of 32 amino acids (SEQ ID NO: 36). Similarly, the reverse complement of BMNI-17 (SEQ ID NO: 37) contains an open reading frame that encodes an amino acid sequence (SEQ ID NO: 38) having a degenerate 32 amino acid repeat (SEQ ID NO: 39).

The reverse complement of BMNI-3 (SEQ ID NO: 40) has an open reading frame which shows homology with the BMNI-4-like genes. The predicted amino acid sequence encoded by this open reading frame is shown in SEQ ID NO: 41. The reverse complement of BMNI-5 (SEQ ID NO: 42) contains a partial copy of a BMNI-3-like sequence and also an open reading frame with some homology to two yeast genes (S. cerevisiae G9365 ORF gene, and S. cerevisiae accession no. U18922). The predicted 5′ and 3′ amino acid sequences encoded by this open reading frame are shown in SEQ ID NO: 43 and 44, respectively. The reverse complement of BMNI-7 (SEQ ID NO: 45) contains an open reading frame encoding the amino acid sequence shown in SEQ ID NO: 46.

A telomeric repeat sequence, which is conserved over a wide range of organisms, was found in five antigens (BMNI-2, BMNI-5, BMNI-6, BMNI-7 and BMNI-16), indicating that many of the isolated genes may have a telomere-proximal location in the genome. BMNI-10 appears to include a double insert, the 3′-most segment having some homology to E. coli aminopeptidase N. In addition, BMNI-7 contains apparently random insertions of hamster DNA. One such insertion has characteristics of a transposible element (i.e. poly A tail and flanked by a direct repeat).

In subsequent studies, two additional B. microti antigens were isolated by screening the B. microti genomic DNA expression library described above with a serum pool from B. microti infected patients that showed low reactivity with recombinant proteins generated from clones BMNI-2-BMNI-17. The determined DNA sequences for these two clones, hereinafter referred to as MN-10 and BMNI-20, are provided in SEQ ID NO: 50 and 51, respectively, with the corresponding predicted amino acid sequences being provided in SEQ ID NO: 52 and 53. MN-10 was found to extend the sequence of BMNI-4 in the 3′ direction and BMNI-20 was found to extend the sequence of BMNI-17 in the 5′ direction.

### EXAMPLE 2

### Synthesis of Synthetic Polypeptides

Polypeptides may be synthesized on a Millipore 9050 peptide synthesizer using FMOC chemistry with HPTU (O-Benzotriazole-N,N,N′,N′-tetramethyluronium hexafluorophosphate) activation. A Gly-Cys-Gly sequence may be attached to the amino terminus of the peptide to provide a method of conjugating or labeling of the peptide. Cleavage of the peptides from the solid support may be carried out using the following cleavage mixture: trifluoroacetic acid:ethanedithiol:thioanisole:water:phenol (40:1:2:2:3). After cleaving for 2 hours, the peptides may be precipitated in cold methyl-t-butyl-ether. The peptide pellets may then be dissolved in water containing 0.1% trifluoroacetic acid (TFA) and lyophilized prior to purification by C18 reverse phase HPLC. A gradient of 0-60% acetonitrile (containing 0.1% TFA) in water (containing 0.1% TFA) may be used to elute the peptides. Following lyophilization of the pure fractions, the peptides may be characterized using electrospray mass spectrometry and by amino acid analysis.

This procedure was used to synthesize two peptides (hereinafter referred to as BABS-1 and BABS-4) made to the repeat region of the isolated B. microti antigen BMNI-3. The sequences of BABS-1 and BABS-4 are shown in SEQ ID NO: 47 and 48, respectively.

### EXAMPLE 3

### Use of Representative Antigens and Peptides for Serodiagnosis of B. microti Infection

A. Diagnostic Properties of Representative Antigens and Peptides as Determined by ELISA

The diagnostic properties of recombinant BMNI-3, BMNI-4, BMNI-6, BMNI-15, MN-10 and BMNI-20, and the BABS-1 and BABS-4 peptides were determined as follows.

Assays were performed in 96 well plates coated overnight at 4° C. with 200 ng antigen/well added in 50 μl of carbonate coating buffer. The plate contents were then removed and the wells were blocked for 2 hours with 200 μl of PBS/1% BSA. After the blocking step, the wells were washed six times with PBS/0.1% Tween 20™. Fifty microliters of sera, diluted 1:100 in PBS/0.1% Tween 20™/0.1% BSA, was then added to each well and incubated for 30 minutes at room temperature. The plates were then washed six times with PBS/0.1% Tween 20™.

The enzyme conjugate (horseradish peroxidase-Protein A, Zymed, San Francisco, Calif.) was then diluted 1:20,000 in PBS/0.1% Tween 20™/0.1% BSA, and 50 μl of the diluted conjugate was added to each well and incubated for 30 minutes at room temperature. Following incubation, the wells were washed six times with PBS/0.1% Tween 20™. 100 μl of tetramethylbenzidine peroxidase substrate (Kirkegaard and Perry Laboratories, Gaithersburg, Md.) was added, undiluted, and incubated for 15 minutes. The reaction was stopped by the addition of 100 μl of 1N H₂SO₄ to each well and the plates were read at 450 nm.

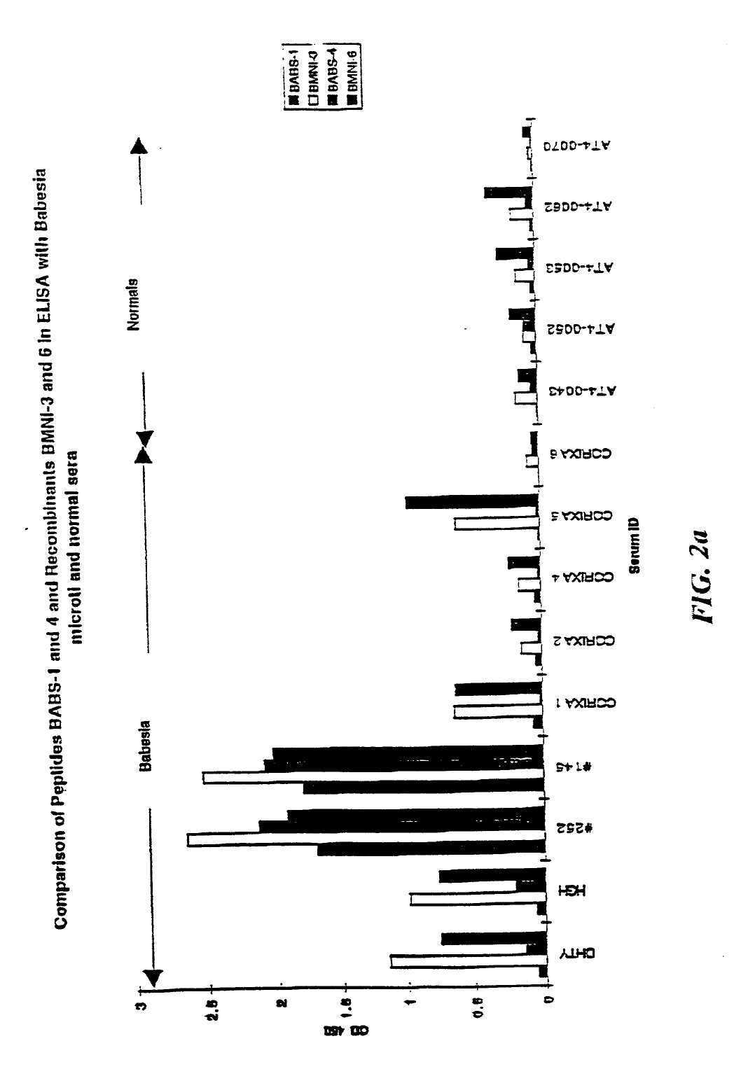

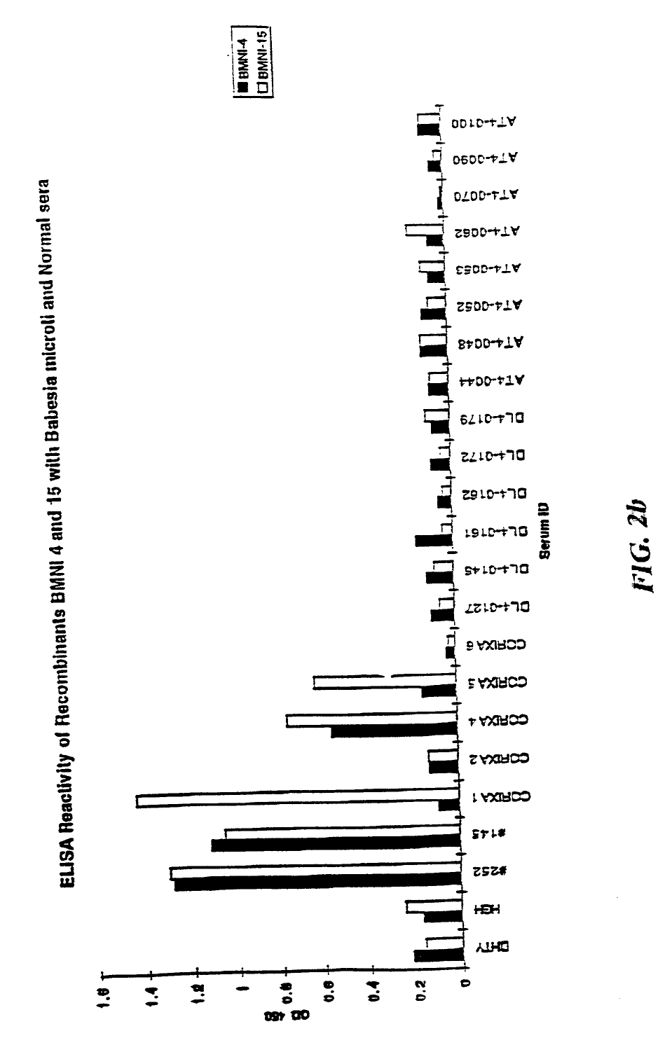

FIG. 2a shows the reactivity of the recombinant BMNI-3 and BMNI-6 antigens and the two peptides BABS-1 and BABS-4 in the ELISA assay. The recombinant antigens and the two peptides were negative in ELISA with all seven samples from normal (B. microti negative) individuals. In contrast, both BMNI-3 and BMNI-6 detected six of the nine B. microti-infected samples, as compared to two out of the nine for the BABS-1 and BABS-4 peptides. This would suggest that BMNI-3 and BMNI-6 may contain other antigenic epitopes in addition to those present in the repeat epitopes in BABS-1 and BABS-4, or that an insufficient number of repeats are available in the peptides to fully express the antigenic epitopes present in the recombinant antigens BMNI-3 and BMNI-6.

FIG. 2b shows the ELISA reactivity of the recombinant antigens BMNI-4 and BMNI-15. Both recombinants were negative with all fifteen samples from normal individuals. BMNI-4 detected four out of nine B. microti-infected samples and BMNI-15 detected six out of nine B. microti-infected samples. Both BMNI-4 and BMNI-15 detected a B. microti-infected sample which was not detected by BMNI-3 or BMNI-6, suggesting that BMNI-4 and BMNI-15 might be complementary to BMNI-3 and BMNI-6 in the ELISA test described herein.

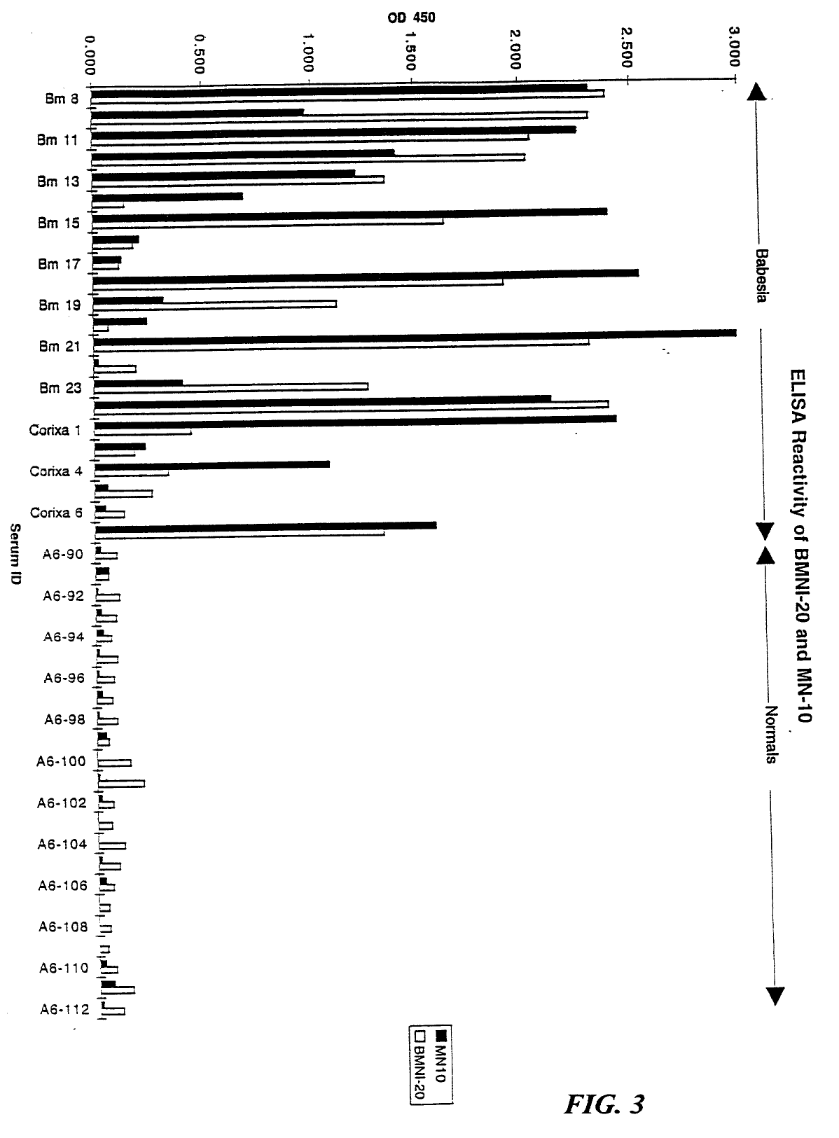

The ELISA reactivity of recombinant MN-10 and BMNI-20 with sera from B. microti-infected patients and from normal donors is shown in FIG. 3. MN-10 and BMNI-20 were found to be reactive with B. microti-infected sera that were not reactive with recombinant BMNI-2 through BMNI-17. Therefore, MN-10 and BMNI-20 may be usefully employed in combination with other B. microti antigens of the present invention for the detection of B. microti infection.

Table 1 shows the reactivity of the recombinant B. microti antigens BMNI-2, BMNI-17, MN-10 and a combination of BMNI-17 and MN-10, as determined by ELISA, with Babesia-positive sera, sera positive for both Babesia and Ehrlichia, sera positive only for Ehrlichia, Lyme disease sera and sera from normal donors. The data indicate a sensitivity of approximately 93% and a specificity in normal donors in excess of 98%. These results indicate that a combination of BMNI-17 and MN-10 is particularly effective in the diagnosis of B. microti infection.

TABLE 1

| | | | | | Normal |
|---|---|---|---|---|---|
| Antigen | Babesia | Babesia/Ehrlichia | Ehrlichia | Lyme | donors |
| BMNI-2 | 27/50 | 2/3 | 1/4 | 0/10 | 1/73 |
| BMNI-17 | 35/50 | 3/3 | 0/4 | 0/10 | 0/86 |
| MN-10 | 37/49 | 3/3 | 0/4 | 1/10 | 1/98 |
| BMNI-17/ | 46/50 | 3/3 | 0/4 | 1/10 | 1/98 |
| MN-10 | | | | | |

B. Diagnostic Properties of Representative Antigens and Peptides as Determined by Western Analysis

Western blot analyses were performed on representative B. microti antigens as follows.

Antigens were induced as pBluescript SK-constructs (Stratagene), with 2 mM IPTG for three hours (T3), after which the resulting proteins from time 0 (T0) and T3 were separated by SDS-PAGE on 15% gels. Separated proteins were then transferred to nitrocellulose and blocked for 1 hr in 0.1% Tween 2™/PBS. Blots were then washed 3 times in 0.1% Tween 2™/PBS and incubated with a B. microti patient serum pool (1:200) for a period of 2 hours. After washing blots in 0.1% Tween 20™/PBS 3 times, immunocomplexes were detected by the addition of Protein A conjugated to ¹²⁵I (1/25000; NEN-Dupont, Billerica, Mass.) followed by exposure to X-ray film (Kodak XAR 5; Eastman Kodak Co., Rochester, N.Y.) at −70° C. for 1 day.

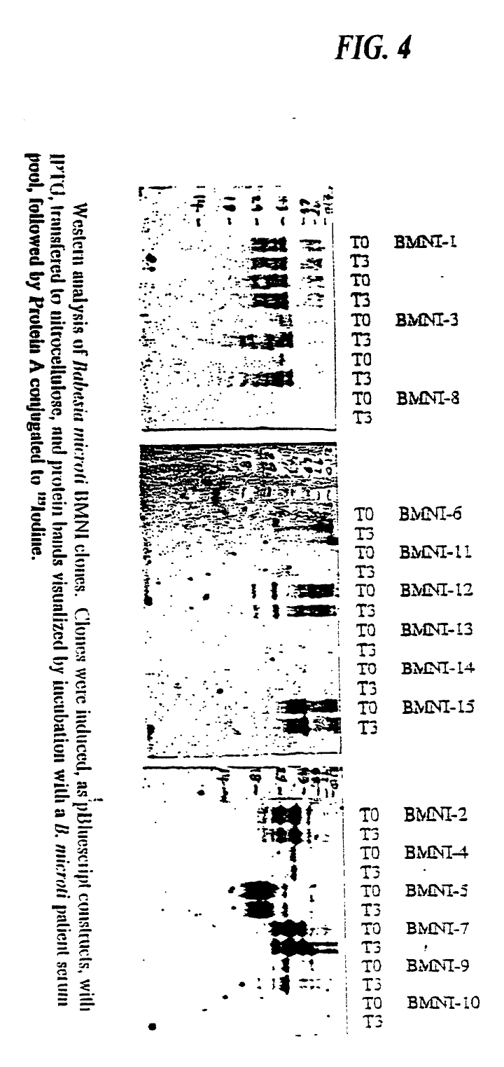

As shown in FIG. 4, resulting bands of reactivity with serum antibody were seen at 43 kDa for BMNI-1, 38 kDa for BMNI-2, 45 kDa for BMNI-3, 37 kDa for BMNI-4, 18 and 20 kDa for BMNI-5, 35 and 43 kDa for BMNI-7, 32 kDa for BMNI-9, 38 kDa for BMNI-11, 30 kDa for BMNI-12, 45 kDa for BMNI-15, and 43 kDa for BMNI-17 (not shown). Antigen BMNI-6, after reengineering as a pET 17b construct (Novagen, Madison, Wis.) showed a band of reactivity at 33 kDa (data not shown). Protein size standards, in kDa (Gibco BRL, Gaithersburg, MB), are shown to the left of the blots.

Western blots were performed on purified BMNI-3, BMNI-2, BMNI-15, BMNI-17 and MN-10 recombinant antigen with a series of patient sera from B. microti patients and from patients with either Lyme disease or ehrlichiosis. Specifically, purified recombinant antigen (4 μg) was separated by SDS-PAGE on 12% gels. Protein was then transferred to nitrocellulose membrane for immunoblot analysis. The membrane was first blocked with PBS containing 1% Tween 20™ for 2 hours. Membranes were then cut into strips and incubated with individual sera (1/500) for two hours. The strips were washed 3 times in PBS/0.1% Tween 20™ containing 0.5 M NaCl prior to incubating with Protein A-horseradish peroxidase conjugate (1/20,000) in PBS/0.1% Tween 20™/0.5 M NaCl for 45 minutes. After further washing three times in PBS/0.1% Tween 20™/0.5 M NaCl, ECL chemiluminescent substrate (Amersham, Arlington Heights, Ill.) was added for 1 min. Strips were then reassembled and exposed to Hyperfilm ECL (Amersham) for 5-30 seconds.

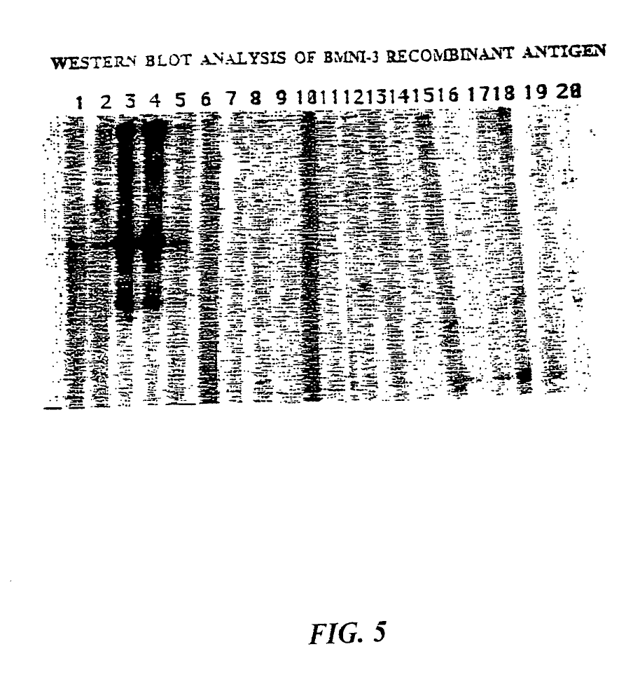

Lanes 1-9 of FIG. 5 show the reactivity of purified recombinant BMNI-3 with sera from nine B. microti-infected patients, of which five were clearly positive and a further two were low positives detectable at higher exposure to the hyperfilm ECL. This correlates with the reactivity as determined by ELISA. In contrast, no immunoreactivity was seen with sera from patients with either ehrlichiosis (lanes 10 and 11) or Lyme disease (lanes 12-14), or with sera from normal individuals (lanes 15-20). A major reactive band appeared at 45 kDa and a small break down band was seen at approximately 25 kDa.

Table 2, below, summarizes the reactivity of the recombinant antigens BMNI-2, BMNI-15, BMNI-17 and MN-10 with B. microti positive sera. No reactivity was seen with Lyme or Ehrlichia-infected sera, with little or no reactivity being seen with normal sera.

TABLE 2

| Sample ID | BMNI-2 | BMNI-15 | BMNI-17 | MN-10 |
|---|---|---|---|---|
| BM8 | ++ | ++ | +++++ | − |
| BM21 | ++ | − | ++++ | ++++ |
| COR4 | ± | ++++ | ++++ | + |
| COR5 | ± | +++ | + | − |
| 252 | ++++ | ++++ | ++++++ | +++ |

− indicates no reactivity

### EXAMPLE 4

### Analysis of Geographic Variation within Antigens

The reactivity of the inventive antigens with sera from B. microti patients, as determined by Western blot, was found to vary with the U.S. location of the patients. Accordingly, geographic variation within the gene encoding the exemplary antigen BMNI-6 was examined as follows.

Two PCR primers, referred to as BMNI-6/5′ and BMNI-6/3′ (SEQ ID NOS: 54 and 55, respectively) were designed based on the region flanking the six amino acid degenerate repeat region of BMNI-6 (SEQ ID NO: 6). These primers were employed to amplify genomic DNA from whole blood obtained from twelve B. microti-infected patients and genomic DNA from whole blood from P. leucopus and hamsters in a Perkin Elmer 480 thermal cycler using the manufacturer's protocol. PCR products were evaluated for size on 2% agarose gels and then Southern blotted and probed with a DIG-labeled oligonucleotide. Positive clones were sequenced using an Applied Biosystems Model 373A or 377 sequencer. RT-PCR was performed on Trizol LS extracted B microti-infected hamster whole blood RNA using the primers described above, and the resulting clones were sequenced as described above.

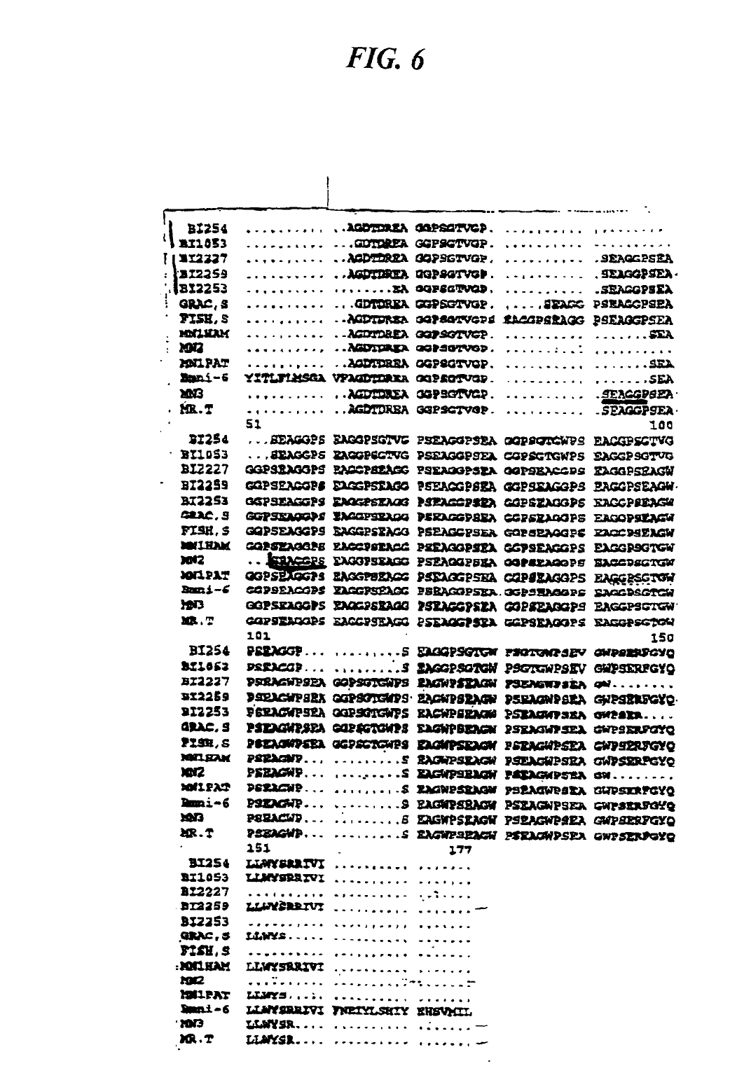

These studies resulted in the isolation of twelve BMNI-6 homologues, referred to hereinafter as BI1254, BI1053, BI2227, BI2259, BI2253, BI2018, RIFS, MN1HAM, MN2, MN1PAT, MN3 and MRT with MN1HAM being obtained from hamster and the other eleven from patients. The determined DNA sequences of these clones are provided in SEQ ID NO: 56-67, respectively, with the corresponding predicted amino acid sequences being provided in SEQ ID NO: 68-79, respectively. Isolates from hamsters had the same sequences as found in the corresponding human blood, suggesting that genetic variation of BMNI-6 does not occur during passage. However, clones from different patients often showed variation in the number and location of the degenerate repeat found within BMNI-6. An alignment of the repeat regions from each of the twelve clones is provided in FIG. 6. Furthermore, strains that were closely related geographically were also closely related at the sequence level. For example, three patients from Nantucket Island, Mass., harbored clones (BI2253, BI2259 and BI2227) that were indistinguishable from each other but distinct from those found in other northeastern or upper Midwestern strains. These results suggest that considerable antigenic diversity exists among isolates of B. microti from the U.S. and that geographic clustering of subtypes exists.

### EXAMPLE 5

### Preparation and Characterization of B. microti Fusion Proteins

A fusion protein containing the B. microti antigens MN-10 and BMNI-17, referred to as BaF-3, was prepared as follows.

MN-10 and BMNI-17 DNA was used to perform PCR using the primers PDM-285 and PDM-286 (SEQ ID NO: 80 and 81); and PDM-283 and PDM-284 (SEQ ID NO: 82 and 83), respectively. In both cases, the DNA amplification was performed using 10 μl of 10× Pfu buffer (Stratagene), 1 μl of 10 mM dNTPs, 2 μl each of the PCR primers at 10 μM concentration, 83 μwater, 1.5 μl Pfu DNA polymerase (Stratagene, La Jolla, Calif.) and 1 μl DNA at 50 ng/μl. Denaturation at 96° C. was performed for 2 min, followed by 40 cycles of 96° C. for 20 sec, 60° C. for 15 sec and 72° C. for 3 min, and lastly by 72° C. for 4 min. The MN-10 and BMNI-17 PCR products were digested with SspI and then ligated using a ligation kit from Panvera (Madison, Wis.). The resulting BaF-3 fusion was PCR amplified using primers PDM 285 and PDM-284 and the same conditions as listed above. This PCR product was then digested with ScaI and EcoRI, and cloned into a modified pET28 vector. The fusion construct was confirmed by sequencing. The expression construct was transformed into BL21 pLys S E. coli (Novagen, Madison, Wis.) for induction and expression.

One of skill in the art will appreciate that the order of the individual antigens within the fusion protein may be changed and that comparable or enhanced activity could be expected provided each of the epitopes is still functionally available. In addition, truncated forms of the proteins containing active epitopes may be used in the construction of fusion proteins.

Although the present invention has been described in some detail by way of illustration and example for purposes of clarity of understanding, changes and modifications can be carried out without departing from the scope of the invention which is intended to be limited only by the scope of the appended claims.

1. An isolated polypeptide comprising an immunogenic portion of a B. microti antigen or a variant thereof, wherein said antigen comprises an amino acid sequence encoded by a DNA sequence selected from the group consisting of: (a) sequences recited in SEQ ID NO: 1-17, 37, 40, 42, 45, 50 and 51; (b) the complements of said sequences; and (c) DNA sequences that hybridize to a sequence of (a) or (b) under moderately stringent conditions.

2. An isolated antigenic epitope of a B. microti antigen comprising the amino acid sequence -X₁-X₂-X₃-X₄-X₅-Ser-, wherein X₁ is Glu or Gly, X₂ is Ala or Thr, X₃ is Gly or Val, X₄ is Trp or Gly and X₅ is Pro or Ser.

3. An isolated antigenic epitope according to claim 2 wherein X₁ is Glu, X₂ is Ala and X₃ is Gly.

4. An isolated antigenic epitope according to claim 2 wherein X₁ is Gly, X₂ is Thr and X₅ is Pro.

5. An isolated polypeptide comprising at least two contiguous antigenic epitopes according to claim 2.

6. An isolated antigenic epitope of a B. microti antigen comprising an amino acid sequence selected from the group consisting of SEQ ID NO: 36 and 39.

7. An isolated polypeptide comprising at least two contiguous antigenic epitopes according to claim 6.

8. An isolated polynucleotide comprising a DNA sequence encoding a polypeptide according to any one of claims 1, 5 and 7.

9. A recombinant expression vector comprising a polynucleotide according to claim 8.

10. A host cell transformed with an expression vector according to claim 9.

11. The host cell of claim 10 wherein the host cell is selected from the group consisting of E. coli, yeast and mammalian cells.

12. A fusion protein comprising at least two polypeptides according to any one of claims 1, 5 and 7.

13. A fusion protein comprising a polypeptide having an amino acid sequence of SEQ ID NO: 32.

14. The fusion protein of claim 13 further comprising a polypeptide having an amino acid sequence of SEQ ID NO: 52.

15. A fusion protein comprising two or more antigenic epitopes according to claim 2 or 6.

16. A fusion protein comprising at least one polypeptide according to any one of claims 1, 5 and 7, and at least one antigenic epitope according to any one of claims 2 and 6.

17. A method for detecting B. microti infection in a patient, comprising:
(a) obtaining a sample from the patient;
(b) contacting the sample with at least one polypeptide comprising an immunogenic portion of a B. microti antigen; and
(c) detecting the presence of antibodies that bind to the polypeptide.

18. A method for detecting B. microti infection in a patient, comprising:
(a) obtaining a sample from the patient;
(b) contacting the sample with at least one antigenic epitope according to any one of claims 2 and 6; and
(c) detecting the presence of antibodies that bind to the antigenic epitope.

19. The method of claim 18 wherein the antigenic epitope is bound to a solid support.

20. The method of claim 19 wherein the solid support comprises nitrocellulose, latex or a plastic material.

21. A method for detecting B. microti infection in a patient, comprising:
(a) obtaining a sample from the patient;
(b) contacting the sample with at least one polypeptide according to any one of claims 1, 5 and 7; and
(c) detecting the presence of antibodies that bind to the polypeptide.

22. A method for detecting B. microti infection in a patient, comprising:
(a) obtaining a sample from the patient;
(b) contacting the sample with at least one polypeptide according to any one of claims 1, 5 and 7, and at least one antigenic epitope according to any one of claims 2 and 6; and
(c) detecting the presence of antibodies that bind to the polypeptide or antigenic epitope.

23. A method for detecting B. microti infection in a patient, comprising:
(a) obtaining a sample from the patient;
(b) contacting the sample with a fusion protein according to any one of claims 12-16; and
(c) detecting the presence of antibodies that bind to the fusion protein.

24. The method of claim 17, 18, 21, 22 or 23 wherein the biological sample is selected from the group consisting of whole blood, serum, plasma, saliva, cerebrospinal fluid and urine.

25. The method of claim 24 wherein the biological sample is whole blood.

26. A method for detecting B. microti infection in a biological sample, comprising:
(a) contacting the sample with at least two oligonucleotide primers in a polymerase chain reaction, wherein at least one of the oligonucleotide primers is specific for a DNA molecule according to claim 8; and
(b) detecting in the sample a DNA sequence that amplifies in the presence of the first and second oligonucleotide primers.

27. The method of claim 26 wherein at least one of the oligonucleotide primers comprises at least about 10 contiguous nucleotides of a DNA molecule according to claim 8.

28. A method for detecting B. microti infection in a biological sample, comprising:
(a) contacting the sample with one or more oligonucleotide probes specific for a DNA molecule according to claim 8; and
(b) detecting in the sample a DNA sequence that hybridizes to the oligonucleotide probe.

29. The method of claim 28 wherein the probe comprises at least about 15 contiguous nucleotides of a DNA molecule according to claim 8.

30. The method of claim 26 or 28 wherein the biological sample is selected from the group consisting of whole blood, sputum, serum, plasma, saliva, cerebrospinal fluid and urine.

31. A method for detecting B. microti infection in a biological sample, comprising:
(a) contacting the biological sample with a binding agent which is capable of binding to a polypeptide comprising an immunogenic portion of a B. microti antigen; and
(b) detecting in the sample a polypeptide that binds to the binding agent, thereby detecting B. microti infection in the biological sample.

32. A method for detecting B. microti infection in a biological sample, comprising:
(a) contacting the biological sample with a binding agent which is capable of binding to a polypeptide according to any one of claims 1, 5 and 7; and
(b) detecting in the sample a polypeptide that binds to the binding agent, thereby detecting B. microti infection in the biological sample.

33. A method of detecting B. microti infection in a biological sample, comprising:
(a) contacting the biological sample with a binding agent which is capable of binding to an antigenic epitope according to any one of claims 2 and 6; and
(b) detecting in the sample an antigenic epitope that binds to the binding agent, thereby detecting B. microti infection in the biological sample.

34. A method of detecting B. microti infection in a biological sample, comprising:
(a) contacting the biological sample with a first binding agent which is capable of binding to a polypeptide according to any one of claims 1, 5 and 7, and a second binding agent which is capable of binding to an antigenic epitope according to any one of claims 2 and 6; and
(b) detecting in the sample a polypeptide that binds to the first binding agent or an antigenic epitope that binds to the second binding agent, thereby detecting B. microti infection in the biological sample.

35. A method of detecting B. microti infection in a biological sample, comprising:
(a) contacting the biological sample with a binding agent which is capable of binding to a fusion protein according to any one of claims 12-16; and
(b) detecting in the sample a polypeptide that binds to the binding agent, thereby detecting B. microti infection in the biological sample.

36. The method of claim 32, 33, 34 or 35 wherein the binding agent is a monoclonal antibody.

37. The method of claim 32, 33, 34 or 35 wherein the binding agent is a polyclonal antibody.

38. A diagnostic kit comprising:
(a) at least one polypeptide comprising an immunogenic portion of a B. microti antigen; and
(b) a detection reagent.

39. A diagnostic kit comprising
(a) at least one polypeptide according to any one of claims 1, 5 and 7; and
(b) a detection reagent.

40. The kit of any one of claims 38 and 39 wherein the polypeptide is immobilized on a solid support.

41. The kit of claim 40 wherein the solid support is selected from the group consisting of nitrocellulose, latex, and plastic materials.

42. A diagnostic kit comprising:
(a) at least one antigenic epitope according to any one of claims 2 and 6; and
(b) a detection reagent.

43. The kit of claim 42 wherein the antigenic epitope is immobilized on a solid support.

44. The kit of claim 43 wherein the solid support is selected from the group consisting of nitrocellulose, latex, and plastic materials.

45. A diagnostic kit comprising:
(a) at least one antigenic epitope according to any one of claims 2 and 6;
(b) at least one polypeptide according to any one of claims 1, 5 and 7; and
(c) a detection reagent.

46. A diagnostic kit comprising:
(a) at least one fusion protein according to any one of claims 12-16; and
(b) a detection reagent.

47. The kit of any one of claims 38, 39, 42, 45 and 46 wherein the detection reagent comprises a reporter group conjugated to a binding agent.

48. The kit of claim 47 wherein the binding agent is selected from the group consisting of anti-immunoglobulins, Protein G, Protein A and lectins.

49. The kit of claim 47 wherein the reporter group is selected from the group consisting of radioisotopes, fluorescent groups, luminescent groups, enzymes, biotin and dye particles.

50. A diagnostic kit comprising at least one polymerase chain reaction primer, the primer being specific for a DNA molecule according to claim 8.

51. The kit of claim 50 wherein the polymerase chain reaction primer comprises at least about 10 contiguous nucleotides of a DNA molecule according to claim 8.

52. A diagnostic kit comprising at least one oligonucleotide probe, the oligonucleotide probe being specific for a DNA molecule according to claim 8.

53. The kit of claim 52 wherein the oligonucleotide probe comprises at least about 15 contiguous nucleotides of a DNA molecule according to claim 8.

54. A monoclonal antibody that binds to a polypeptide according to any one of claims 1, 5 and 7.

55. A monoclonal antibody that binds to an antigenic epitope according to any one of claims 2 and 6.

56. A polyclonal antibody that binds to a polypeptide according to any one of claims 1, 5 and 7.

57. A polyclonal antibody that binds to an antigenic epitope according to any one of claims 2 and 6.

58. A pharmaceutical composition comprising at least one polypeptide according to any one of claims 1, 5 and 7, and a physiologically acceptable carrier.

59. A pharmaceutical composition comprising at least one DNA molecule according to claim 8 and a physiologically acceptable carrier.

60. A pharmaceutical composition comprising at least one antigenic epitope according to any one of claims 2 and 6, and a physiologically acceptable carrier.

61. A vaccine comprising at least one polypeptide according to any one of claims 1, 5 and 7, and a non-specific immune response enhancer.

62. A vaccine comprising at least one DNA molecule according to claim 8 and a non-specific immune response enhancer.

63. A vaccine comprising at least one antigenic epitope according to any one of claims 2 and 6, and a non-specific immune response enhancer.

64. The vaccine of any one of claims 61-63 wherein the non-specific immune response enhancer is an adjuvant.

65. A method for inducing protective immunity in a patient, comprising administering to a patient a pharmaceutical composition according to any one of claims 58-60.

66. A method for inducing protective immunity in a patient, comprising administering to a patient a vaccine according to any one of claims 61-63.


SEQUENCE LISTING (83 sequences)

SEQ ID NO: 1 - DNA, 792 bp, Babesia microti
cactctttttaatgagcggtgctgtctttgcaagtgataccgatcccgaagctggtgggcctagtgaagctggtgggcctagtggaactgttgggcccagtgaagctggtgggcctagtgaagctggtgggcctagtggaactggttggcctagtgaagctggtgggcctagtgaagctggtgggcctagtgaagctggtgggcctagtgaagctggtgggcctagtggaactggttggcctagtggaactggttggcctagtgaagctggttggtctagtgaacgatttggatatcagcttcttccgtattctagaagaatagttatatttaatgaagtttgtttatcttatatatacaaacatagtgttatgatattggaacgagatagggtgaacgatggtcataaagactacattgaagaaaaaaccaaggagaagaataaattgaaaaaagaattggaaaaatgttttcctgaacaatattcccttatgaagaaagaagaattggctagaatatttgataatgcatccactatctcttcaaaatataagttattggttgatgaaatatcaaacaaggcctatggtacattggaaggtccagctgctgataattttgaccatttccgtaatatatggaagtctattgtacttaaagatatgtttatatattgtgacttattattacaacatttaatctataaattctattatgacaataccgttaatgatatcaagaaaaattttgacgaatccaaatctaaagctttagttttgagggataagatca

SEQ ID NO: 2 - DNA, 2732 bp, Babesia microti
aaaccctaaaccctaaaccctaaaccctaaaccctaaacccctaaaccctaaaccctaaaccctaaaccctaaaccctaaaaccctaaaccctaaaccctaaaccctaaaccctaaaccctaaaccctaaaccctaaaccctaaaccctaaaccctaaaccctaaaccctaaaccctaaaccctaaaccctaaaccctaaaccctaaaccctaaacccctaaaccctaaaccctaaaccctaaaccctaaaccctaaaccctaaaccctaaaccctaaaccctaaaccctaaaccctaaaccctaaaccctaaaccctaaaccctaaaccctaaaaccctaaaccctaaaccctaaaccctaaaccctaaaccctaaacccctaaaccctaaaccctaaaccctaaaccctaaaccctaaacccctaaaccctaaacccctaaaccctaaaccctaaaccctaaaccctaaaccctaaaccctaaaccctaaaccctaaaccctaaaccctaaacccctaaaccctaaaccctaaaccctaaaccctaaaccctaaaccctaaaccctaaaccctaaccctaaccctaaccctaaccctaacctagccttcattgacgtctatccccaatcttagaaaaatcttcaaatcgattctagaataactggaaacaattatcagaaattgtataactgcttattagcttattagcttattagttaggatgtatgcacattgatgacaactagatgcagcaccacaatcactaccacgtaccaatcatataccaataatgtactaataatgtaccaataactatggtttataaagatggtgtcatttaaatcaatattagttccttatattacactctttttaatgagcggtgctgtctttgcaagtgataccgatcccgaagctggtgggcctagtgaagctggtgggcctagtggaactgttgggcccagtgaagctggtgggcctagtgaagctggtgggcctagtggaactgttgggcccagtgaagctggtgggcctagtgaagctggtgggcctagtggaactggttggcctagtgaagctggtgggcctagtgaagctggtgggcctagtggaactgttgggcccagtgaagctggtgggcctagtgaagctggtgggcctagtggaactggttggcctagtgaagctggtgggcctagtgaagctggtgggcctagtgaagctggtgggcctagtgaagctggtgggcctagtggaactggttggcctagtggaactggttggcctagtgaagctggttggtctagtgaacgatttggatatcagcttcttccgtattctagaagaatagttatatttaatgaagtttgtttatcttatatatacaaacatagtgttatgatattggaacgagatagggtgaacgatggtcataaagactacattgaagaaaaaaccaaggagaagaataaattgaaaaaagaattggaaaaatgttttcctgaacaatattcccttatgaagaaagaagaattggctagaatatttgataatgcatccactatctcttcaaaatataagttattggttgatgaaatatcaaacaaggcctatggtacattggaaggtccagctgctgataattttgaccatttccgtaatatatggaagtctattgtacttaaagatatgtttatatattgtgacttattattacaacatttaatctataaattctattatgacaataccgttaatgatatcaagaaaaattttgacgaatcctggacacagacattaaaagaataagcctgcttgggggtttctgggcatctcttcatgagtgccagtcacacaactcttctgtgagccttctacaataaggactttgtgtgcttcgatatttttttagactaaagtgaactctctcctccacctttggcttcagttagttatttcaaatggcaaaagttattaaaaattccagtgtggaaactggcttaaccaacaggaaaggggttttgaggtcgcatcactaagcatcaagtttaacaccaacatgcctggaggattggcttagccggttgctagggcaggcctgtggcagggttcttatcccagctattaacgctcccttcccactcctccaagtcctgcaagtcctggatacagtgaaatgtaattgcatatcccatatcctttgctagtatcaaatggataaaacccaaaatggagtcataccaaatgatctcatgtatacaatacctgaatagtcttgaactgatgcactgttagatagtatgcacttactcttcagctattcatagtgtgcctctgcacagtgatggaaaagaggagcactgggggagctcggttttcaagggacaaaggagaataagacacacaaagaaatccaaggtagagcagagaaaggatggagacacagaaggtttgcaggaacaggaagcgaaggatgctccagtctgagggggaggggaaagagagcctcttgagtagccagcacctgaacttggcctggaagcttggtggataaggcaggataaaggaggtgtggcctctttggtatcctcccattgataaaggagctccctgacccttcactagaccatcatcagtcctatggttcttagaccaatagaacacaatggaattgatttgttccactttccaggttaagacttacagtcagggaagtttgtttttcttgcc

SEQ ID NO: 3 - DNA, 2430 bp, Babesia microti
aactagatgcagcaccacaatcactaccacgtaccaatcatataccaataatgtactaataatgtaccaataactatggtttataaagatggtgtcatttaaatcaatattagttccttatattacactctttttaatgagcggtgctgtctttgcaagtgataccgatcccgaagctggtgggcctagtgaagctggtgggcctagtggaactgttgggcccagtgaagctggtgggcctagtgaagctggtgggcctagtggaactggttggcctagtgaagctggtgggcctagtgaagctggtgggcctagtgaagctggtgggcctagtgaagctggtgggcctagtggaactggttggcctagtggaactggttggcctagtgaagctggttggtctagtgaacgatttggatatcagcttcttccgtattctagaagaatagttatatttaatgaagtttgtttatcttatatatacaaacatagtgttatgatattggaacgagatagggtgaacgatggtcataaagactacattgaagaaaaaaccaaggagaagaataaattgaaaaaagaattggaaaaatgttttcctgaacaatattcccttatgaagaaagaagaattggctagaatatttgataatgcatccactatctcttcaaaatataagttattggttgatgaaatatcaaacaaggcctatggtacattggaaggtccagctgctgataattttgaccatttccgtaatatatggaagtctattgtacttaaagatatgtttatatattgtgacttattattacaacatttaatctataaattctattatgacaataccgttaatgatatcaagaaaaattttgacgaatccaaatctaaagctttagttttgagggataagatcactaaaaaggatggagattataacactcattttgaggacatgattaaggagttgaatagtgcagcagaagaatttaataaaattgttgacatcatgatttccaacattggggattatgatgagtatgacagtattgcaagtttcaaaccatttctttcaatgatcaccgaaatcactaaaatcaccaaagtttctaatgtaataattcctggaattaaggcactaactttaaccgtttttttaatatttattacaaaatagatgtaataccagatgtatacattattatatattacaaaatttacacattatttatgtatgaacgaacgaacatctcagtcttaaatgaagaaattgggataaatatggaaatagattaaagtaacatgagaaagatgaatataatattagaatatgaaatttaacagaaataaaatgaagtaaaagagtgtattttgtaataatttataataaattagtatacaatgattatattacagatgactattgattattgtatcaattaaatattgattattaatgatatcatatatgtatatgttaatgattgatttgttatacgttgtgaatatgttatataatgacatactataataattaatataatgtagaggatattttttttaatagtatttaatgaatattatagttataattataataatgtagataaaaatgacattaatttgaatgtttaaattgaaatgtatgtaaaaatatgtatttataatctgaattgattaataatataatattctacaattaattatttttgtaattataataattgattatattaatctttgaattattataaataatattatacttcattaaattatttcacataaatttccaaattattatcctttatcttaatgttatccaattttacacatctttcttcattacaatatttttttactaatcctgtatgctcatattcatattctttagaaatataacgaaaattagatgtaacttcgccacttacaagtaaactaccatcaatataataataatgaataccattcatgtccgtatattctttatattttttatcatattttattttgtgattattccattcatttgtatcattattcaatgagagaaataatagcagaaagatccttctatagaaacataaaattcaattaatactggattattatgtttgcaagtatagatgtttaaatcaataacactaccagttggtaatttagcattgtcatcaaattcaattatataatcagaaattttgattttatcaattttattcggatgtgataatttattttgttctgattcatcgatcatgtatacaaatactattgttaaaggttccctatccttataattaaagtggccaataagattggcattaattacattagtagtgtgtgtatttgtaatagtatcattagtggtactgacagttgttataggttttgatttccataatgaaacatcatttttatctacacaataca

SEQ ID NO: 4 - DNA, 1991 bp, Babesia microti
aatgtacaagatcaaaatttctgattatataattgaatttgatgacaatgctaaattaccaactgataatgttattggtatatccatctatacttgtgaacacaataatccagtattaattgaattttatgtttctaaaaaaggatcaatctgctattatttctactcaatgaataatgatacaaataaatggaataatcacaaaataaaatatgacaaaagatttaatgaacatactgacatgaatggtattcattattattatattgatggtagtttacttgcgagtggcgaagttacatctaattttcgttatatttctaaagaatatgaatatgagcatacagaattagcaaaagagcattgcaagaaagaaaaatgtgtaaatgtggataacattgaggataataatttgaaaatatatgcgaaacagtttaaatctgtagttactactccagctgatgtagcgggtgtgtcagatggattttttatacgtggccaaaatcttggtgctgtgggcagtgtaaatgaacaacctaatactgttggtatgagtttagaacaattcatcaagaacgagctttattcttttagtaatgaaatttatcatacaatatctagtcaaatcagtaattctttcttaataatgatgtctgatgcaattgttaaacatgataactatattttaaaaaaagaaggtgaaggctgtgaacaaatctacaattatgaggaatttatagaaaagttgaggggtgctagaagtgaggggaataatatgtttcaggaagctctgataaggtttaggaatgctagtagtgaagaaatggttaatgctgcaagttatctatccgccgcccttttcagatataaggaatttgatgatgaattattcaaaaaggccaacgataattttggacgcgatgatggatatgattttgattatataaatacaaagaaagagttagttatacttgccagtgtgttggatggtttggatttaataatggaacgtttgatcgaaaatttcagtgatgtcaataatacagatgatattaagaaggcatttgacgaatgcaaatctaatgctattatattgaagaaaaagatacttgacaatgatgaagattataagattaattttagggaaatggtgaatgaagtaacatgtgcaaacacaaaatttgaagccctaaatgatttgataatttccgactgtgagaaaaaaggtattaagataaacagagatgtgatttcaagctacaaattgcttctttccacaatcacctatattgttggagctggagttgaagctgtaactgttagtgtgtctgctacatctaatggaactgaatctggtggagctggtagtggaactggaactagtgtgtctgctacatctactttaactggtaatggtggaactgaatctggtggaacagctggaactactacgtctagtggaacttggtttggaaaatgaaaaattagctctagaaacactttattgttaatttttaaaaacctattgaaaaatcagattgtaaaacataattccacttctaaccatgctatgatttaactaatcaggacaaaaagaaagcataatcaacattattcattcagtgatggtgacataattcagagaatgtggcaattgcctcttgaagaccagagttccatccacaggacccacatggttaaaggagagagctaactcctgaaagttgtcctctgactaacacattcaacttttgagtgtctcatttatgtgttggcttctgtctaatgtgggaaaatcattaagggctcttaaatcagatcctcattctctctattaataaactatgtgataacatccttcagctatgaaaatgtcaggagagagtcaggaaaatggaagatattgttcaggacttaactaggtggtggcacacagttcctttacacagattcctcaggacaagttttaggtgaggttttgatctatcctg

SEQ ID NO: 5 - DNA, 1271 bp, Babesia microti
ttcactaggccaaccagcttcactaggccaaccagcttcactaggccaaccagcttcactaggccaaccagcttcactaggccaaccagcttcactaggccaaccagttccactaggcccaccagcttcactaggcccaccagcttcactaggcccaccagcttcactaggccaaccagttccactaggcccaccagcttcactaggcccaccagcttcactaggcccaccagcttcactaggcccaccagcttcactaggcccaccagcttcactaggcccaccagcttcactaggcccaccagcttcactaggcccaccagcttcactaggcccaacagttccactaggcccaccagcttcgcgatcggtatcacctgcaaagacagcaccgctcattaaaaagagtgtaatataaggaactaatattgatttaaatgacaccatctttataaaccatagttattggtacattattagtacattattggtatatgattggtacgtggtagtgattgtggtgctgcatctagttgtcatcaatgtgcatacatcctaactaataagctaataagctaataagcagttatacaatttctgataattgcttccagttattctagaatcgatttgaagatttttctaagattggggatagacgtcaatgaaggctaggttagggttagggttagggttagggttagggtttagggtttagggtttagggtttagggtttagggttagggtttagggtttagggtttagggtttaggctcccaagttgtcccgtgaaagggccgtgtctttgataaattttgccgtcctgtacgtttcctttctagaatgcacaaaaacaagaatttggcagctagaaacatcgttaatcacctcttggtagagaatttcgttgattgcgttgaaacgtttgatagccttcttctccttcacgccataatacacctgctccaagggcacaggcctaaagtggctgccaaagtagaaaagccctcggtctagattaacagtgagaaatctagccacgtcttcgtagtttggaagcgtggccgatagaccaactagccttacgcgttcgggcctctgactcaggcgggccacaatagcctccagcactggacccctagtgtcatggagtaggtgtatttcatcaattataaccaatctaagccgctcaagcaggggctcattgcctgttttacgtgtaactacgtcaaacttctctggcgtagttacaattatatgcgttttctca

SEQ ID NO: 6 - DNA, 1821 bp, Babesia microti
taaaccctaaacccctaaaccctaaaccctaaaccctaaaccctaaaccctaaacccctaaaccctaaaccctaaaccctaaaccctaaaccctaaccctaaaccctaaaccctaaaccctaaaccctaaaccctaaccctaaccctaaccctaaccctaacctagccttcattgacgtctatccccaatcttagaaaaatcttcaaatcgattctagaataactggaagcaattatcagaaattgtataactgcttattagcttattagcttattagttaggatgtatgcacattgatgacaactagatgcagcaccacaatcactaccacgtaccaatcatataccaataatgtactaataatgtaccaataactatggtttataaagatggtgtcatttaaatcaatattagttccttatattacactctttttaatgagcggtgctgtctttgcaggtgataccgatcgcgaagctggtgggcctagtggaactgttgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtggaactggttggcctagtgaagctggttggcctagtgaagctggttggcctagtgaagctggttggcctagtgaagctggttggcctagtgaagctggttggcctagtgaacgatttggatatcagcttctttggtattctagaagaatagttatatttaatgaaatttatttatctcatatatacgaacatagtgttatgatattggaacgagatagggtgaacgatggtcataaagactacattgaagaaaaaaccaaggagaagaataaattgaaaaaagaattggaaaaatgttttcctgaacaatattcccttatgaagaaagaagaattggctagaataattgataatgcatccactatctcttcaaaatataagttattggttgatgaaatatccaacaaagcctatggtacattggaaggtccagctgctgatgattttgaccatttccgtaatatatggaagtctattgtacctaaaaatatgtttctatattgtgacttattattaaaacatttaatccgtaaattctattgtgacaataccattaatgatatcaagaaaaattttgacgacatagagaaattgggctgttttcaagctagaagcttcctccctgttaactaatgtattcatggtgccagaaggtgctatgcaggttgctagggaatcaaattcatcaatagtcctgcccaagagtagtgtgttaactggcggtgcaagatgtgccctttgatgcagtagtggcatgcttgtttgtggggtaacccagtgctttctgattgaggtctactccacaggaggaatagatacctgcttctgtaaacttggtcaaaacttatgactgcacatgaagacagagtggaaaagacctgaaaacacacacggggtcaggactgaggaagacagggttagtattagagagatttggggaaaaaaagagttagcaaatatagagtgtgatagtctaatggggggatgaatggtatcaaaatgaattatttatatgtataaaactgacaattttttaattgtgaaaaggaatgcaatccgacccatctgggggaattctagctagcatcagtgagagaagaggcaaggtgttaggaaatcgtgcagaacatgctcatccaggctttatttctccatttacatctagag

SEQ ID NO: 7 - DNA, 4223 bp, Babesia microti
catcacaattattggctgttacatcactatagtgctgtatgtaaaaaattataaagtgtgacatcattataatgcaatatgacatcacaattatatactgtgacttcactatcttgcactttaacatcacaattatacattgtgacatcaatatactgcactatgacatcacgattattgactgtgacatcaatacattctctatgaacacagttatacactctgacatcactagcttgcactgtgacatgacaattaaaaactgtgacatcaatataatggactgtgacctacaattattcactgtgaaaccacaacactgcaattgtgtataattgggatgggtactgatctgctgcccgaggctcaatagattacctaggcctcctcactgacacccacattcagggggtcttgatcagtcccatgatggattcccaggctgatgcctgggattcaagagttaacctttgtctggtcagctctttctgggggttaaacggattaaatgttttaataataagtcacaatatagaaacatatttttaggtacaatagacttccatatattacggaaatggtcaaaatcatcagcagctggaccttccaatgtaccataggctttgttggatatttcatcaaccaataacttatattttgaagagatagtggatgcattatcaattattctagccaattcttctttcttcataagggaatattgttcaggaaaacatttttccaattcttttttcaatttattcttctccttggttttttcttcaatgtagtctttatgaccatcgttcaccctatctcgttccaatatcataacactatgttcgtatatatgagataaataaatttcattaaatataactattcttctagaataccaaagaagctgatatccaaatcgttcactaggccaaccagcttcactaggccaaccagcttcactaggccaaccagcttcactaggccaaccagcttcactaggccaaccagcttcactaggccaaccagcttcactaggcccaccagcttcactaggcccaccagcttcactaggcccaccagcttcactaggcccaacagttccactaggcccaccagcttcactaggcccaccagcttcactaggcccaccagcttcactaggcccaccagcttcactaggcccaccagcttcactaggcccaccagcttcactaggcccaccagcttcactaggcccaacagttccactaggcccaccagcttcgcgatcggtatcacctgcaaagacagcaccgctcattaaaaagagtgtaatataaggaactaatattgatttaaatgacaccatctttataaaccatagttattggtacattattagtacattattggtatatgattggtacgtggtagtgattgtggtgctgcatctagttgtcatcaatgtgcatacatcctaactaataagctaataagctaataagcagttatacaatttctgataattgcttccagttattctagaatcgatttgaagatttttctaagattggggatagacgtcaatgaaggctaggttagggttagggttagggttagggttagggtttagggtttagggtttagggtttagggtttagggttagggtttagggtttagggtttagggtttagggtttaggggtttagggtttagggtttagggtttagggtttagggtttagggtttagggaaggctgagaaccactgacttagactttccaagactttgtcatcttatgacttgccggttgcctcgtttctccacacagcaacctatgttctctcttattacagtttctgtgggacatgtcatgcttccagcttcgagaatggaagcctattgtcttaatgggtgagcaaagtgggcccattcattaatcacagactaatccaaaaggaaatgtgacacctgacctaagtccgaccaataggagccaggaaagctcacttctggaattgtgacttagatatcacggatgcatacagactctttttcctgctgaaacaaatggtgaggacctgtccacccttgtgggaagcttgcagtgtaagattctaatccatattggggaaataaggctgagaagagagagttccaggccttgtgacagaatctaatccctggataaagtctctctttttacaaagaacatcagtgttgcaagctccaaattcctgttcttactttcttgagtctgttttctttatgtataacccaaagcactttaactgacacagctgtgaagtgagaatatttcatagaaatcctattgttttgatgtcttctaaaaaagaaaaaaagcaatgatctgtaacattttttaacttaaataattagattgatttaagtgacatcaaaacatctggaaaatggtgtggacacaaattcactagagagccatattttttgctaactaattgagaaattaatcactggcaagtctttggtaaaagtatcacctcagtcatgatctctcctgccttcatgacattttcctcattggtgtgaggatgctattctgctttctatgtgaccaggaaatagtgctgtcttctgtctagttatgatttaggttgtacaccaggttttcacatatgttccctaacgtctgtagtaggaccagggactggttggcttcaagttgttggatatggttaccttaagtcattcatgtacaggaactcatttgagatgataggaaatgaagtgaaagattttcttgcccctgttaagtaagataaaaaggattgttatgatggggcaggagcagatctatttccaataaacagaatttgaagtgtttgtgtgatattcagatacctcattgtcatttgaatgaattactcctgctctcagtgaagatgtctaagctgcaaataagaaatggagagcgctgtcagaagtcagatggaattgagaataggggcctggctgcaatctgtggagactgcctaaagcagctagataagaaactagcagctggggagagaaagatcgaatttagtcggcctgttttatattttcttataaaaaataactgcttcgaaatgtttgagaagatagaggcaatgagcagaaagttgttccttaaatcagttatagaatgaacacatacacgggcactcagatcaagccatgctgagcttgagacaccgggtgacgcgtgacttgtttattcccaggctgcaaaggagagtaaatgaagtaacgggaaggcccggtgtggtaggcacactcctgcctggcaccatctgctgcttttgtccctgttactccttgttcctttccctccttttctccctcccttcctccctccctctctccctccttcacacttctgtctttatttcctcctgggagttaattggtggtagcccctctgtgctgttctttcgggggtgcctttaatttcgacaatacaatgccatccatgggggcattttatatacagtaataattgtcattgatgtggccataaggtacttttttgtggtacccttcttgaacagaacagacacagaagggcgtgcgtgcgtgcgtgcgtgcgtgcgtgcgtgcgtgtgtgcgtgtgtgcgtgcgtgtgtgcgtgtgtgcgtgcgtgcgtgtgtgcgtgcgtgcgtgtgtgcgtgtgtgtgtgtgtgtgtgtgtgtgtgtgtgtgtgtgtgtgtgtgttgggatggggtggggagcgctagcttcctacttgttgtagggtgatgaggttttatatagtctgtttctgagacagttaccaaatccagctgggttactttttttttggttttttatgagacagggtttctctgtattgttttggaggctgtcggtccagcctggtctcgaactcacagagatccgcctgcctctgcctcccgagtgctgggattaaaggtgtgcgccaccaccgcccggccccagctgggttacttatcactcagtggatctttctcttttctttgtaagaagaactttgcattgtgggtcgtcatggaagaacacttggaaaggtaccctttctgccccacccgtttattgaatgagtcttttttttttttaattaaatagcagaactttggggaaagatttagaaaaggcccttttcatattataatacgaggtataggatggtttaagataagagactttttgttagctgttatcagttgagaaaggcacgag

SEQ ID NO: 8 - DNA, 2287 bp, Babesia microti
ttataaacatatctaaatattttaataataatgatgaaatttaacatagataagataatattaatcaatttaatagtattattgaatcgaaatgtagtgtattgtgtggatacaaataatagttcattaattgaatcacaaccagtaacaactaacattgacactgataatacaattacaacaaataaatacactggtactataattaatgccaatattgttgagtaccgtgaatttgaggatgaacctttaacaatagggtttagatacactatagataaatcacaacaaaataaattatcacatccaaataaaattgataaaatcaaattttctgattatataattgaatttgatgacaatgctaaattaccaactgataatgttatttgtatatccatctatacttgcaagcataataatccagtattaattagattctcatgttctatagaaaaatattactaccattacttctactcaatgaataatgatacaaataaatggaataatcacaaattaaaatatgataaaacatacaatgaatatactgacaataatggtgttaattattataaaatctattatagtgataaacagaattcccctactaatggaaatgaatatgaggatgtagcattagcaagaatacattgtaatgaagaaagatgtgcaaatgtaaaggtagataaaattaaatataagaatttggaaatttatgtgaaacagttaggtactataattaatgccaatattgttgagtaccttgtatttgaggatgaacctttaacaatagggtttagatacactatagataaatcacaacaaaatgaattatcacatccaaataaaatttataaaatcaaattttctgattatataattgaatttgatgatgatgctaaattaacaacaattggtactgttgaagatataaccatctatacttgcaagcataataatccagtattaattagattctcatgttctatagaaaaatattactactattacttctactcaatgaataataatacaaataaatggaataatcacaacttaaaatatgataatagattcaaagaacatagtgacaagaatggtattaattattatgaaatctcagctttcaaatggagtttctcttgttttttcgttaataaatatgagcataaagaattagcaagaatacattgtaatgaagaaagatgtgcaaatgtaaaggtagataaaattaaatataagaatttggaaatttatgtgaaacagttaggtactataattaatgccaatattgttgagtaccttgtatttgaggatgaacctttaacaatagggtttagatacactatagataaatcacaacaaaatgaattatcacatccaaataaaatttataaaatcaaattttctgattatataattgaatttgatgatgatgctaaattaacaacaattggtactgttgaagatataaccatctatacttgcaagcataataatccagtattaattagattctcatgttctatagaaaaatattactactattacttctactcaatgaataataatacaaataaatggaataatcacaacttaaaatatgataatagattcaaagaacatagtgacaagaatggtattaattattatgaaatctcagctttcaaatggagtttctcttgttttttcgttaataaatatgagcataaagaattagcaagaatacattgtaatgaagaaaaatgtgtaaatgtaaaggtagataacattgggaataaaaatttggaaatttatgtgaaataatttaatgaagtataatattatttataataattcaaagattaatataattaattattataattacaaaaataattaattgtagaatattatattattaatcaattcagattataaatacatatttttacatacatttcaatttaaacattcaaattaatgtcatttttatctacattattataattataactataatattcattaaatactatttaaaaaaatatcctctacattatatcaatcaatataatatacaattatataatatattcacaatgtataacaatcaaccctaacatgtacatacataatatcattactaatcaatatttaattaataaaatatttaatagtcatctgtaatataatcattgtatactaatttattataaattattacaaaatacactcttttacttcattttatttctgttaaatttcatattctaatattatattcatctttctcatgttactt

SEQ ID NO: 9 - DNA, 2784 bp, Babesia microti
cactgctttcgcagcgtttcttgcttttgggaatatctcacctgtactttctgctggtggtagtggtggtaatggtggtaatggtggtggtcatcaagagcaaaataatgctaatgatagtagtaatcccaccggagccggtggacaacccaataacgaaagtaagaaaaaggcagtaaaacttgacttggacctcatgaaagaaacaaagaatgtttgcaccactgttaatactaaactagtcggaaaagcaaagagcaaattaaacaaattagaaggtgaatcccataaggagtatgtagctgagaaaacgaaggagatagatgagaaaaataagaaatttaacgagaatcttgttaaaatagagaaaaagaagaaaattaaggttcctgccgatactggtgctgaagtggatgctgttgatgatggtgttgcgggtgcactatccgatttatcctccgatatctccgctattaagactctcaccgacgatgtatccgagaaggtttctgaaaacttgaaagatgatgaggccagtgcaacagaacacactgatataaaagaaaaagccaccctgcttcaagagtcttgcaacggaattggcactatcctagataagttggccgaatatttaaataatgatacaactcaaaatatcaagaaagaatttgatgaacgcaagaagaatctcacctctttgaagacaaaggtagaaaataaggatgaagattatgttgatgttaccatgacatcaaaaacagatctgataatacactgtttaacttgcacaaacgatgcacacggactgtttgatttcgaatcgaagagcttgataaaacaaacctttaaattgaggtccaaagatgaaggtgaactctgctaatttagattttagatgggccatgtatatgttaaacagcaagattcatcttatagaaagcagtttgatcgataacttcaccttggataatccatccgcatacgaaattttacgcgtttcttataactcaaatgaatttcaagtacaatcaccgcagaacattaacaatgaaatggaatcttcaacgcccgaatccaatatcatttgggttgtacatagtgatgttataatgaaaaggttcaactgtaaaaatcgcaaatctctcagtactcattcactcactgaaaatgatattctcaagtttggccgtatagaactctctgttaaatgtataattatgggcgcaggtatcactgcatctgatcttaatctaaagggattggggtttattagtccagataaacaatcaactaatgtatgtaactattttgaagatatgcatgaatcttatcatattcttgatacacaaagggcctcggattgtgtatcagatgatggcgctgatattgatatatccaacttcgacatggtccaagacggtaacataaattctgttgacgctgattctgaaacatgtatggcaaactctggcgtaacggtcaataatactgaaaatgttagtaatagtgagaattttggaaaattaaaatcattggtaagcaccaccactcctttgtgccgtatttgcctgtgtggtgaatcagaccctgggccactagtaaccccttgcaattgcaaggggtccctaaattatgtccatcttgaatgcctaaggacttggattaaagggcggttgtcaattgtgaaggatgatgatgcttcctttttctggaaagagctatcatgtgagctatgcgggaagccgtatccatcggtcctacaagtagatgatacagagactaatttgatggatataaaaaaaccggatgcaccatatgtggtattggaaatgagatcaaattctggtgatgggtgtttcgttgtttctgtagctaaaaataaggcgattattggacgggggcatgaaagtgacgttaggttgagtgatatttcagtgtcacgaatgcatgcttctttggaattggatggtggaaaagtagtgatacatgaccagcaatctaagtttggtacactcgttagggccaaagcgcctttttcaatgcctataaagggtcccatctgtctacaggtaagcattttctttttgaacttgaaaatatctactcatagtctaaccatggagaggggcatggaacatgtccttctctaatatttccaaaaaggatctatgcctgataaccttggtattgaaggtggctttctcaaagtgagacattccatttctgttgttggagctatcctatctgaggttagtgttctggtaaacattcctagaaaactcataaagcagaaatctgtgtgtatactaaattgcacagagaactccacgtgtgtgctagacttcacagagaactctgtgtgtgtgctaaactgcatagagaagaacatgttgagtgcatcatggttgagggaaattgctttatataaaagatttattttcctaaggtaacttaggattaatttttctgaaagcttagttttggtgagcacaattgtgatctttgtttctcagatggtcgggaaggcactcccagaaagcaggtggatacacactacactgcatgctacactctgtagactaggagtatcgttttcacacttatgaaatagtcaccatgctgggcacaaatatctttttatacaccatatattgttcatgttcaggtccacatttcaatttgtatgtgaaaagcatccggggctgtctgataaacacatagaaatgaaggaaacagtgtatgtaactgaagccttcagtcctttgcaatttctttgattcttag

SEQ ID NO: 10 - DNA, 3701 bp, Babesia microti
acctatttataatatagtatattactggtttgttttaaatcgaaaaaatgtattgtatttaagaatgaaattatttatttatcatgattatcatatttctaaatattaaaatctagtaacggttgcttgaatatttatttaaattatatgtagtagtattaaaatgtgttatatataagtagtgttctaaatcatcattagtaatattgtataaattaattgtaaaaattgcgatactacaattaatcaacaattaaaatatatcagtatagataatttaaataaataattagataagatcttaaggattaaatgacgaatttagaatgataaataatcatcataggcatttgttataatatcattaattatattcatgtggttataattataaaagtatatatagttttgtaattgtaatgatataaaattagaacagatataattaataattcaaatattatattaattttattatatatgattattattgatatttatataattacatattgttattgtatcatttaatgattatatatcaatatccatatatatatataataattgaattataattaaattaattggcatattacatttataataatatattattagtcaatatgacatcatattatattatccatcatgattgtgaatgtaactagaacattgattattatattaaatcacatattaatactgattataataatatcattgataatctaataatatagtattatctctaataatattgtattatctctaatattatggtataatagatactgtgaaaataaattcaactggagataaggaaaccattttgtatagatattttatacaaattattatgaaataatctaaataaatgacaaaaaatcgattatacaaatcacattaatgacaaacaaacttgtatacatatattgattaacattacaaaactaaattataatatttagattgataattgttataatacttaacaatattctactttttaatataattttttattcaataatatactctttcatattttgtactattttatataatcatatatattatataattatatatatttgataattgaatatatcaataatgatgatatacatgaatatgcatatataccccatataatgttattatatttagtgcttacattattaattataaatatatttaaataattaaataataatgaaaattaacatagacaatataatattaatcaatttgataatattattgaatcgtaatgtagtatattgtgtggataaaaatgatgtttcattatggaaatcaaaacctataacaactgtcagtaccactaatgatactattacaaataaatacactagtactgtaattaatgccaattttgctagctaccgtgaatttgaggatagggaacctttaacaataggatttgaatacatgatcgataaatcacaacaagataaattatcacatccaaataaaattgataaaatcaaaatttctgattatataattgaatttgatgacaatgctaaattaccaactggtagtgttaatgatatatccatcattacttgcaagcataataatccagtattaattagattctcatgtttaatagaaggatctatctgctattatttctacttattgaataatgatacaaataaatggaataatcacaaattaaaatatgataaaacatacaatgaacatactgacaataatggtattaattattataaaatcgattatagtgaatctacagaacctactaccgaatctactacctgtttttgttttcgcaaaaaaaatcataaatctgagcgtaaagaattagaaaattataaatatgagggtacagaattagcaagaatacattgtaataaagggaaatgtgtaaaattgggtgacattaagataaaggataagaatttggaaatttatgtgaaacagttaatgtctgtaaatactccagtaaattttgacaaccctacatcgattaatctaccaactgtcagtactaccaatgatactattacaaataaatacactggtactataattaatgccaatattgttgagtactgtgaatttgaggatgaacctttaacaatagggtttagatacactatagataaatcacaacaaaataaattatcacatccaaataaaattgataaaatcaaattttttgattatataattgaatttgatgatgatgttaaattaccaacaattggtactgtcaatattatatatatctatacttgcgagcataataatccagtattagttgaatttatagtttctatagaagaatcttactacttttacttctactcaatgaataataatacaaataaatggaataatcacaaattaaaatatgataaaagattcaaaaaatatactaagaatggtattaattgttatgaatatgtacttcgtaaatgcagttcttatactcgtaaaaatgaatatgagcataaagaattagcaagaatacattgtaatgaagaaaaatgtgtaaatgtaaaggtagataacattgagaaaaagaatttggaaatttatgtaaaataatttaacgaagtgtaatatgtaaaatagtttaatgaagtataatattatttaaaataattcaaaatttcagaaattaatataattaattattataaatacaaaataattaattacaaatgtgtattgttagttatttcagattgtaaatacatattttacatacatttttattaaaactttcaaattaatattttcatttttataagcattattataattatatactataattatcagtcatcaaataatatccaaagttatcctctacattatatcaatcatacagtatacaattatataaaatattaacaacatataacaaccaacattaatatatacataatatctttattaatcaatatttaatcaatacaataattaatagttaactaactatacacatagtgtatactaaattattataaattatatgttataattacaaaaacgtcatttacttattttatttcagttatgtttcatagtctaatttagatttggtgaaacgcatctggctgatgtgctggtgagcaagcagttccacgaagcaaacaatatgactgatgcgctggcggcgctttctgcggcggttgccgcacagctgccttgccgtgacgcgctgatgcaggagtacgacgacaagtggcatcagaacggtctggtgatggataaatggtttatcctgcaagccaccagcccggcggcgaatgtgctggagacggtgcgcggcctgttgcagcatcgctcatttaccatgagcaaccccgaaccgtattcgttcgttgattggcgcgtttgcgggcagcaatccggcagcgttccatgccgaagatggcagcggttacctgttcctggtggaaatgcttaccgacctcaacagccgtaacccgcaggtggcttcacgtctgattgaaccgctgattcgcctgaaacgttacgatgccaaacgtcaggagaaaatgcgcgcggcgctggaacagttgaaagggctggaaaatctctctggcgatctgtacgagaagataactaaagcactggcttgataaataaccgaatggcggcaatagcgccgccattcggggaatttacccctgttttct

SEQ ID NO: 11 - DNA, 1287 bp, Babesia microti
ctcgtgccgctcgtgccgattattataaatatttagttgatgaatatagttctcccagggaggaaagagaattagcaagagtacattgtaatgaagaaaaatgtgtaaaattggatggcattaagtttaaggataagaatttggaaatttatgtgaaacagttaatgtctgtaaatactccagttgtatttgacaacaatacattgattaatccaactagcagcagtggtgccactgatgacataacatatgaattatcggtggaatcacaacctgtaccaactaacattgacacaggtaataatattacaacaaatacatcaaataataatctaattaaagctaaatttctttataattttaatcttcctggtaaaccttcaacaggactatttgaatacactatagataaatcagaacaaaataaattatcacatccaaataaaattgataaaatcaaattttctgattatataattgaatttgatgatgatgctaaattaccaacaattggtactgtcaatattatatccatcattacttgcaagcataataatccagtattagttgaatttatagtttctacagaaatatattgctactacaattacttctactcaatgaataataatacaaataaatggaataatcacaaattaaaatatgataaaagatataaagaagaatatacagatgataatggtattaattattataaattaaatgatagtgaacctactgaatctacagaatctactacctgtttttgttttcgcaaaaaaaatcataaatatgaaaatgagcgtacagcattagcaaaagaacattgcaatgaagaaagatgtgtaaaggtagataacattaaggataataatttggaaatttatctaaaataatttaacgaagtataatattatttataataattcaaaatttcagaaattaatataattaattattataaatacaaaataattaattacaaatgtgtattgttagttatttcagattgtaaatacatattttacatacatttttattaaaactttcaaattaatattttcatttttataagcattattataattatatactataattatcagtcatcaaataatatccaaagttatcctctacattatatcaatcatacagtatacaattatataaaatattaacaacatataacaaccaacattaatatatacataatatctttattaatcaatatttaatcaatacaataattaatagttaactaactatacacatagtgtatactaaatt

SEQ ID NO: 12 - DNA, 572 bp, Babesia microti
cttcattgacgtctatccccaatcttagaaaaatcttcaaatcgattctagaataactggaaacaattatcagaaattgtataactgcttattagcttattagcttattagttaggatgtatgcacattgatgacaactagatgcagcaccacaatcactaccacgtaccaatcatataccaataatgtactaataatgtaccaataactatggtttataaagatggtgtcatttaaatcaatattagttccttatattacactctttttaatgagcggtgctgtctttgcaagtgataccgatcccgaagctggtgggcctagtgaagctggtgggcctagtgaagctggtgggcctagtggaactgttgggcccagtgaagctggtgggcctagtgaagctggtgggcctagtggaactggttggcctagtgaagctggtgggcctagtgaagctggtgggcctagtggaactggttggcctagtgaagctggttggtctagtgaacgatttggatatcagcttcttccgtattctagaagaatagttacatttaatgaagtttgtttat

SEQ ID NO: 13 - DNA, 2338 bp, Babesia microti
ctcgtgccgaatcttagaaaaatcttcaaatcgattctagaataactggaaacaattatcagaaattgtataactgcttattagcttattagcttattagttaggatgtatgcacattgatgacaactagatgcagcaccacaatcactaccacgtaccaatcatataccaataatgtactaataatgtaccaataactatggtttataaagatggtgtcatttaaatcaatattagttccttatattacactctttttaatgagcggtgctgtctttgcaagtgataccgatcccgaagctggtgggcctagtggaactgttgggcccagtgaagctggtgggcctagtgaagctggtgggcctagtggaactggttggcctagtgaagctggtgggcctagtgaagctggtgggcctagtggaactggttggcctagtgaagctggttggtctagtgaacgatttggatatcagcttcttccgtattctagaagaatagttacatttaatgaagtttgtttatcttatatatacaaacatagtgttatgatattggaacgagatagggtgaacgatggtcataaagactacattgaagaaaaaaccaaggagaagaataaattgaaaaaagaattggaaaaatgttttcctgaacaatattcccttatgaagaaagaagaattggctagaatatttgataatgcatccactatctcttcaaaatataagttattggttgatgaaatatcaaacaaggcctatggtacattggaaggtccagctgctgataattttgaccatttccgtaatatatggaagtctattgtacttaaagatatgtttatatattgtgacttattattacaacatttaatctataaattctattatgacaataccattaatgatatcaagaaaaattttgacgaatccaaatctaaagctttagttttgagggataagatcactaaaaaggacgtgtatgtaaatgatcactaaacgggctccacatatctattactggggtagatattataagttatggataagtaaatttatggcgatagattccaacaaatttgtggttagtagcgacaatgattatggctagtgtgtggagtacttatgagtgaatgattgtagtggtggctagcagtgagtatagttaggtaatccctacacacccatttaaataagatgcaaatagcatttaaattgacatatattgtgtgtatgtccacgtttattgcgtttccatgacgtatctgctgaggtgtgtcttgtgtatctaagtaccagacacagcacttaaattgttatgggcatgacgatggatgttaaaggtttatacactccaaaggcacgttcttctgctagggaaacgagggacaagttcgattttgctatacaaagcaagtttcactccctggactttacactggatgactttgatataggtgcattcgtggtaaacctcaaaatttactcagggcgatggtgcccatgggcaggtttttttggcaagggaacgacgtaccggttttatttgcgtgttaaaatgcatttttaaatcacaacttgtgaagtaattgcctaataatcacacagaaatggacaggaagctattttcaagcgggaaatcgaattgcacgggcatctgagacatccaaacatagcatggtatgtacatatttatccagcttgtatacctggttcactagccctactatgatattcatagtgatggaatattgttacaatggcgatctatttaattatatgtcaaaacatggccaactgagtgaagaaagggtatcagagtatacagatatttacatagaattttgttcgaagtcatttgggccattagaagctgccacgacaaacgcatagcgcacttggatattaaaccagtaaggttctatgttacagaggagaatatattattggaccatgaaaacaggtgtaaattggcggactttggattctctgcacacatagggcatttgtaccgctcaaacggagtgctcatcatcgtggcacgcatggtaacacgcaattwatggcagattattggtctccggagcagtgtgccaaacatttgggtctggggttgaagtatggggagtatgatgaacaaagcgacatatgggcgttgggcatattggcagttgaattgtttattggataccctccatttggatctactactgaagagcccaacaatgtgattatgaacagaatccacacttaccactggaccaaacatgtacttttatctattacgcagatttttgaaatgaagagggaaaaacatctactctcgtcgacgcctg

SEQ ID NO: 14 - DNA, 729 bp, Babesia microti
ttgcctggaccttctctgtcctagaattacaggaattctcttatactgtttaatacaaaacacttggaagaatttcaccaattgcatatgaaacatggaatccaagagaccaaaatttaaaaccttgaaatagaagcacttatgccaatattggaaattacttagtgaagtgatccaaagtactgatttggtcagaagacatcaccagggcactagctggcctagtgacctgagtatttgtgaaagctgattttaatgttgagaacatgaaggaagcagtattgaggtaatggaatcttgtagattatagtagaagccaactgagaccaagaaatgtacggtaggaatgaaataaggtcttgggtggtcattgcatggagctgtgaaagtgaagcgttgttggggtatagattcgcaagtcttggggcatgactatgtggggttaccaaggttaggttaactgaggtggaaagatccactctaaatgggggagttaccatttcatgtgctgggatcccagagatgtcaaaggagaaaataagctattgaataagagcatctatatcccttgcttcttggctatggatgttatgtgactagtcatctcttagtcttaccttcaccattataacaagattttctagaactttgggttaaattaaatcctttattcctcacgttgctgtcttagttactttcctgttgctttgataaagcattctggccaag

SEQ ID NO: 15 - DNA, 1448 bp, Babesia microti
acatgttgacttttggaaatatacgttttcataatataaatctcccaccattttcattgggcataattcactcgattacggtagaaaaggcgattaactctgaagattttgacggaatacaaacacttttacaagtgtctatcattgctagttacggtccatctggcgattacagtagttttgtgttcactccagttgtaacagcagacaccaacgttttttacaaattagagacggatttcaaacttgatgttgatgttattactaagacatcactagaattgcccacaagtgttcctggctttcactacaccgaaactatttaccaaggcacagaattgtcaaaatttagcaagcctcagtgcaaacttaacgatcctcctattacaacaggatcggggttgcaaataatacatgatggtttgaataattcgacaattataaccaacaaagaagttaatgtggatggaacagatttagttttttttgaattgctccctccatcggatggcattcccaccttgcgatcaaaattatttcccgtcctgaaatcaattccaatgatatctaccggggttaatgaattactgttggaagtactcgagaacccctctttccctagtgcaattagcaattacaccggactgacaggccgacttaacaaattacttacagttttagacggtattgttgatagcgccattagtgtcaagactacagaaactgtccctgacgacgcagaaacttctatttcttcattgaaatcattgataaaggcaatacgagataatattactaccactcgaaacgaagttaccaaagatgatgtttatgcattgaagaaggccctcacttgtctaacgacacacctaatatatcattcaaaagtagatggtatatcattcgacatgctgggaacacaaaaaaataaatctagcccactaggcaagatcggaacgtctatggacgatattatagccatgttttcgaatcccaatatgtatcttgtgaaggtggcgtacttgcaagccattgaacacatttttctcatatcaaccaaatacaatgatatatttgattacaccattgattttagtaagcgtgaagctactgattctggatcatttaccgatatattgctcggaaacaaggtgaaggaatctttgtcatttattgagggtttgatttctgacataaaatctcactcattgaaagctggggttacaggaggtatatcaagttcatcattatttgatgaaatcttcgacgagttaaatttggatcaagcaacaattagaacccttgttgcaccattagattggccacttatctcagacaaaagcctccacccttcactgaagatggttgtggtcctgccaggatttttcatagttccttaataacatgacatttcatagtcccttcagtcctgatgacaagacggtgaa

SEQ ID NO: 16 - DNA, 1350 bp, Babesia microti
gcctaagcccaaatgggatttaagcaggaggggataaaacagatgacctccaccatgccctactaactctaagctaaggaaatccagcctgctggctatttacctgctttcctcgaagtgaaaggccagagtcacccccaatctttcccaaaagattgaagtcactctctccatgccggcaaaggtagatggtgcgaggctggacatggatattcataaggtagtagacaattttactctggatgtagtcctggactctgttgaccagaaatctctggcctacattaatcaccttgatgaagacagatccctaggacagagtagaaagagcaattttatggtcagaaaatctgaaactaggagtgtggcaagcaagggggcaaggctatcagcacctagtgacaatcccagcacttagaaggcttagctggaaggggcttaggtttgaccctgactcaagacaaatgaacatatgaaaagtatggggagaatgatctgtgtattgactggtagggcctcatcagctattccttctctccctgtcactgccatctcgtgccgaattcggcacgagctcgtgccgaaaccctaaaccctaaacccctaaaccctaaaccctaaaccctaaaccctaaaccctaaaccctaaaccctaaaccctaaacccctaaacccctaaaccctaaaccctaaaccctaaaccctaaaccctaaaccctaaaccctaaccctaaccctaaccctaaccctaacctagccttcattgacgtctatccccaatcttagaagaatcttcaaatcgattctagaataactggaaacaattatcagaaattgtataactgcttattagcttattagcttattagttaggatgtatgcacattgatgacaactagatgcagcaccacaatcactaccacgtaccaatcatataccaataatgtactaataatgtaccaataactatggtttataaagatggtgtcatttaaatcaatattagttccttatattacactctttttaatgagcggtgctgtctttgcaagtgataccgatcccgaagctggtgggcctagtgaagctggtgggcctagtggaactgttgggcccagtgaagctggtgggcctagtgaagctggtgggcctagtggaactggttggcctagtgaagctggtgggcctagtgaagctggtgggcctagtgaagctggtgggcctagtgaagctggtgggcctagtggaactggttggcctagtggaactggttggcctagtgaagctggttggtctagtgaacgatttggatatcagcttcttccgtattctagaagaatagttatattt

SEQ ID NO: 17 - DNA, 1820 bp, Babesia microti
ggaaagccttaaacatgcatgggaataatgaaatagtaaaaattgcagccatggcaatgtaataatgagtggatgtttcagtcttgaggctctttaacaagagtgttgtcttgtagtcaaagacaaagtgattcgtcatgccgcattcgcagccaccatcatcatcaggcgacgacgggtctctttcattatcctcgggcttattattgcaaccatgacacccttctttacaaaagtctttttttttcagcggtgtctgagtattatgcgattttattccagccttcccacttttattcttattgagattgccatgctcttcttcatgagcgtcacttgtttcctgcggtgtctgagtatcatacgattttattccagcatttccacttttattcttattgattttgtcatgcccttcttcacactcttcacatatttcttgcgttgtctgagtatcatgcgattttctttcagccttctcacttttattcgtattgattttgtcatgcccttcttcatgagcgtcacttgtttcctgcggtgtctgagtatcatacgattttattccagcatttccacttttattcttattgattttgtcatgcccttcttcacactcttcacatatttcttgcgttgtctgagtatcatacgattttattccagcatttccacttttattcttattgattttgtcatgcccttcttcacactcttcacatatttcttgcgttgtctgagtatcatgcgattttctttcagccttctcacttttattcgtattgggtttgccatgcccttctttacgctcttcatatatttcttgtgccgttagtctcagtaagttgtcaagctcttcatatatttcttgcggtgtctgagtatcatgcgattttctttcagtcttctcacttttattcgtattgagtttgccattcccttcttcatgatcgtcacttgtttcttgcgccgttagtctcattaagttgtcaagctcttcatcatctattgaatggtatggagctgtatcttcccagggtggttgaattatgtcattctcgccgattttaaatgatggttcttcatcatttatatcagatgccatgtctgagtggtgccctaatctagagaattggtgtggtaccccctcatccaaactttcgggcaacaccctggtatcagaatccatttgttcgagcggctcactatcgcaagcgtcttgtggattgatgttatcatgttcctggatttcaacatgtacagattctgaatccgcattgggttctggaatatagttggtaactacatttgtttctagagaagtatcattcttatattaattcatctaagatctgtgcttctttgtttctacacatacagggtgtctcttttcccaacataatatctgtaaattcttcccagaagcagaaccttgttggtaccagacagcatcgggtctctgtgagtttctattcaggcaacaggtgtattctgtttgccagtccaagtgcatcctgtattctagtactggcttactaccccaagcaaatcactggcatcaacatctagcactgagtgaagcatgatctcttctacaaggtgtttttccattgtgttgtaagcccgtatacaaggctgttcccactcaacaatgaagagacctcttagcatgaatggccagatgtctgttctttaaattaaatcaatatgttttgctcaatatgtcagacttgtttgtggtggagccaaaattggaggtcccatcgagatttggagaaacttgaaatgaatgcaaaagatggtgggggctactcgtgccg

SEQ ID NO: 18 - protein, 263 aa, Babesia microti
LFLMSGAVFASDTDPEAGGPSEAGGPSGTVGPSEAGGPSEAGGPSGTGWPSEAGGPSEAGGPSEAGGPSEAGGPSGTGWPSGTGWPSEAGWSSERFGYQLLPYSRRIVIFNEVCLSYIYKHSVMILERDRVNDGHKDYIEEKTKEKNKLKKELEKCFPEQYSLMKKEELARIFDNASTISSKYKLLVDEISNKAYGTLEGPAADNFDHFRNIWKSIVLKDMFIYCDLLLQHLIYKFYYDNTVNDIKKNFDESKSKALVLRDKI

SEQ ID NO: 19 - protein, 310 aa, Babesia microti
MSGAVFASDTDPEAGGPSEAGGPSGTVGPSEAGGPSEAGGPSGTVGPSEAGGPSEAGGPSGTGWPSEAGGPSEAGGPSGTVGPSEAGGPSEAGGPSGTGWPSEAGGPSEAGGPSEAGGPSEAGGPSGTGWPSGTGWPSEAGWSSERFGYQLLPYSRRIVIFNEVCLSYIYKHSVMILERDRVNDGHKDYIEEKTKEKNKLKKELEKCFPEQYSLMKKEELARIFDNASTISSKYKLLVDEISNKAYGTLEGPAADNFDHFRNIWKSIVLKDMFIYCDLLLQHLIYKFYYDNTVNDIKKNFDESWTQTLKE

SEQ ID NO: 20 - protein, 367 aa, Babesia microti
LWFIKMVSFKSILVPYITLFLMSGAVFASDTDPEAGGPSEAGGPSGTVGPSEAGGPSEAGGPSGTGWPSEAGGPSEAGGPSEAGGPSEAGGPSGTGWPSGTGWPSEAGWSSERFGYQLLPYSRRIVIFNEVCLSYIYKHSVMILERDRVNDGHKDYIEEKTKEKNKLKKELEKCFPEQYSLMKKEELARIFDNASTISSKYKLLVDEISNKAYGTLEGPAADNFDHFRNIWKSIVLKDMFIYCDLLLQHLIYKFYYDNTVNDIKKNFDESKSKALVLRDKITKKDGDYNTHFEDMIKELNSAAEEFNKIVDIMISNIGDYDEYDSIASFKPFLSMITEITKITKVSNVIIPGIKALTLTVFLIFITK

SEQ ID NO: 21 - protein, 492 aa, Babesia microti
MYKIKISDYIIEFDDNAKLPTDNVIGISIYTCEHNNPVLIEFYVSKKGSICYYFYSMNNDTNKWNNHKIKYDKRFNEHTDMNGIHYYYIDGSLLASGEVTSNFRYISKEYEYEHTELAKEHCKKEKCVNVDNIEDNNLKIYAKQFKSVVTTPADVAGVSDGFFIRGQNLGAVGSVNEQPNTVGMSLEQFIKNELYSFSNEIYHTISSQISNSFLIMMSDAIVKHDNYILKKEGEGCEQIYNYEEFIEKLRGARSEGNNMFQEALIRFRNASSEEMVNAASYLSAALFRYKEFDDELFKKANDNFGRDDGYDFDYINTKKELVILASVLDGLDLIMERLIENFSDVNNTDDIKKAFDECKSNAIILKKKILDNDEDYKINFREMVNEVTCANTKFEALNDLIISDCEKKGIKINRDVISSYKLLLSTITYIVGAGVEAVTVSVSATSNGTESGGAGSGTGTSVSATSTLTGNGGTESGGTAGTTTSSGTWFGK

SEQ ID NO: 22 - protein, 138 aa, Babesia microti
SLGQPASLGQPASLGQPASLGQPASLGQPASLGQPVPLGPPASLGPPASLGPPASLGQPVPLGPPASLGPPASLGPPASLGPPASLGPPASLGPPASLGPPASLGPPASLGPTVPLGPPASRSVSPAKTAPLIKKSVI

SEQ ID NO: 23 - protein, 303 aa, Babesia microti
LWFIKMVSFKSILVPYITLFLMSGAVFAGDTDREAGGPSGTVGPSEAGGPSEAGGPSEAGGPSEAGGPSEAGGPSEAGGPSEAGGPSEAGGPSGTGWPSEAGWPSEAGWPSEAGWPSEAGWPSEAGWPSERFGYQLLWYSRRIVIFNEIYLSHIYEHSVMILERDRVNDGHKDYIEEKTKEKNKLKKELEKCFPEQYSLMKKEELARIIDNASTISSKYKLLVDEISNKAYGTLEGPAADDFDHFRNIWKSIVPKNMFLYCDLLLKHLIRKFYCDNTINDIKKNFDDIEKLGCFQARSFLPVN

SEQ ID NO: 24 - protein, 592 aa, Babesia microti
MMKFNIDKIILINLIVLLNRNVVYCVDTNNSSLIESQPVTTNIDTDNTITTNKYTGTIINANIVEYREFEDEPLTIGFRYTIDKSQQNKLSHPNKIDKIKFSDYIIEFDDNAKLPTDNVICISIYTCKHNNPVLIRFSCSIEKYYYHYFYSMNNDTNKWNNHKLKYDKTYNEYTDNNGVNYYKIYYSDKQNSPTNGNEYEDVALARIHCNEERCANVKVDKIKYKNLEIYVKQLGTIINANIVEYLVFEDEPLTIGFRYTIDKSQQNELSHPNKIYKIKFSDYIIEFDDDAKLTTIGTVEDITIYTCKHNNPVLIRFSCSIEKYYYYYFYSMNNNTNKWNNHNLKYDNRFKEHSDKNGINYYEISAFKWSFSCFFVNKYEHKELARIHCNEERCANVKVDKIKYKNLEIYVKQLGTIINANIVEYLVFEDEPLTIGFRYTIDKSQQNELSHPNKIYKIKFSDYIIEFDDDAKLTTIGTVEDITIYTCKHNNPVLIRFSCSIEKYYYYYFYSMNNNTNKWNNHNLKYDNRFKEHSDKNGINYYEISAFKWSFSCFFVNKYEHKELARIHCNEEKCVNVKVDNIGNKNLEIYVK

SEQ ID NO: 25 - protein, 463 aa, Babesia microti
IIMKINIDNIILINLIILLNRNVVYCVDKNDVSLWKSKPITTVSTTNDTITNKYTSTVINANFASYREFEDREPLTIGFEYMIDKSQQDKLSHPNKIDKIKISDYIIEFDDNAKLPTGSVNDISIITCKHNNPVLIRFSCLIEGSICYYFYLLNNDTNKWNNHKLKYDKTYNEHTDNNGINYYKIDYSESTEPTTESTTCFCFRKKNHKSERKELENYKYEGTELARIHCNKGKCVKLGDIKIKDKNLEIYVKQLMSVNTPVNFDNPTSINLPTVSTTNDTITNKYTGTIINANIVEYCEFEDEPLTIGFRYTIDKSQQNKLSHPNKIDKIKFFDYIIEFDDDVKLPTIGTVNIIYIYTCEHNNPVLVEFIVSIEESYYFYFYSMNNNTNKWNNHKLKYDKRFKKYTKNGINCYEYVLRKCSSYTRKNEYEHKELARIHCNEEKCVNVKVDNIEKKNLEIYVK

SEQ ID NO: 26 - protein, 297 aa, Babesia microti
RAARADYYKYLVDEYSSPREERELARVHCNEEKCVKLDGIKFKDKNLEIYVKQLMSVNTPVVFDNNTLINPTSSSGATDDITYELSVESQPVPTNIDTGNNITTNTSNNNLIKAKFLYNFNLPGKPSTGLFEYTIDKSEQNKLSHPNKIDKIKFSDYIIEFDDDAKLPTIGTVNIISIITCKHNNPVLVEFIVSTEIYCYYNYFYSMNNNTNKWNNHKLKYDKRYKEEYTDDNGINYYKLNDSEPTESTESTTCFCFRKKNHKYENERTALAKEHCNEERCVKVDNIKDNNLEIYLK

SEQ ID NO: 27 - protein, 121 aa, Babesia microti
LWFIKMVSFKSILVPYITLFLMSGAVFASDTDPEAGGPSEAGGPSEAGGPSGTVGPSEAGGPSEAGGPSGTGWPSEAGGPSEAGGPSGTGWPSEAGWSSERFGYQLLPYSRRIVTFNEVCL

SEQ ID NO: 28 - protein, 267 aa, Babesia microti
LWFIKMVSFKSILVPYITLFLMSGAVFASDTDPEAGGPSGTVGPSEAGGPSEAGGPSGTGWPSEAGGPSEAGGPSGTGWPSEAGWSSERFGYQLLPYSRRIVTFNEVCLSYIYKHSVMILERDRVNDGHKDYIEEKTKEKNKLKKELEKCFPEQYSLMKKEELARIFDNASTISSKYKLLVDEISNKAYGTLEGPAADNFDHFRNIWKSIVLKDMFIYCDLLLQHLIYKFYYDNTINDIKKNFDESKSKALVLRDKITKKDVYVNDH

SEQ ID NO: 29 - protein, 16 aa, Babesia microti
AWTFSVLELQEFSYTV

SEQ ID NO: 30 - protein, 465 aa, Babesia microti
MLTFGNIRFHNINLPPFSLGIIHSITVEKAINSEDFDGIQTLLQVSIIASYGPSGDYSSFVFTPVVTADTNVFYKLETDFKLDVDVITKTSLELPTSVPGFHYTETIYQGTELSKFSKPQCKLNDPPITTGSGLQIIHDGLNNSTIITNKEVNVDGTDLVFFELLPPSDGIPTLRSKLFPVLKSIPMISTGVNELLLEVLENPSFPSAISNYTGLTGRLNKLLTVLDGIVDSAISVKTTETVPDDAETSISSLKSLIKAIRDNITTTRNEVTKDDVYALKKALTCLTTHLIYHSKVDGISFDMLGTQKNKSSPLGKIGTSMDDIIAMFSNPNMYLVKVAYLQAIEHIFLISTKYNDIFDYTIDFSKREATDSGSFTDILLGNKVKESLSFIEGLISDIKSHSLKAGVTGGISSSSLFDEIFDELNLDQATIRTLVAPLDWPLISDKSLHPSLKMVVVLPGFFIVP

SEQ ID NO: 31 - protein, 128 aa, Babesia microti
LWFIKMVSFKSILVPYITLFLMSGAVFASDTDPEAGGPSEAGGPSGTVGPSEAGGPSEAGGPSGTGWPSEAGGPSEAGGPSEAGGPSEAGGPSGTGWPSGTGWPSEAGWSSERFGYQLLPYSRRIVIF

SEQ ID NO: 32 - protein, 245 aa, Babesia microti
QECCLVVKDKVIRHAAFAATIIIRRRRVSFIILGLIIATMTPFFTKVFFFQRCLSIMRFYSSLPTFILIEIAMLFFMSVTCFLRCLSIIRFYSSISTFILIDFVMPFFTLFTYFLRCLSIMRFSFSLLTFIRIDFVMPFFMSVTCFLRCLSIIRFYSSISTFILIDFVMPFFTLFTYFLRCLSIIRFYSSISTFILIDFVMPFFTLFTYFLRCLSIMRFSFSLLTFIRIGFAMPFFTLFIYFLCR

SEQ ID NO: 33 - protein, 293 aa, Babesia microti
TAFAAFLAFGNISPVLSAGGSGGNGGNGGGHQEQNNANDSSNPTGAGGQPNNESKKKAVKLDLDLMKETKNVCTTVNTKLVGKAKSKLNKLEGESHKEYVAEKTKEIDEKNKKFNENLVKIEKKKKIKVPADTGAEVDAVDDGVAGALSDLSSDISAIKTLTDDVSEKVSENLKDDEASATEHTDIKEKATLLQESCNGIGTILDKLAEYLNNDTTQNIKKEFDERKKNLTSLKTKVENKDEDYVDVTMTSKTDLIIHCLTCTNDAHGLFDFESKSLIKQTFKLRSKDEGELC

SEQ ID NO: 34 - protein, 431 aa, Babesia microti
GPKMKVNSANLDFRWAMYMLNSKIHLIESSLIDNFTLDNPSAYEILRVSYNSNEFQVQSPQNINNEMESSTPESNIIWVVHSDVIMKRFNCKNRKSLSTHSLTENDILKFGRIELSVKCIIMGAGITASDLNLKGLGFISPDKQSTNVCNYFEDMHESYHILDTQRASDCVSDDGADIDISNFDMVQDGNINSVDADSETCMANSGVTVNNTENVSNSENFGKLKSLVSTTTPLCRICLCGESDPGPLVTPCNCKGSLNYVHLECLRTWIKGRLSIVKDDDASFFWKELSCELCGKPYPSVLQVDDTETNLMDIKKPDAPYVVLEMRSNSGDGCFVVSVAKNKAIIGRGHESDVRLSDISVSRMHASLELDGGKVVIHDQQSKFGTLVRAKAPFSMPIKGPICLQVSIFFLNLKISTHSLTMERGMEHVLL

SEQ ID NO: 35 - protein, 6 aa, Babesia microti
  VARIANT (1)...(1): Xaa = Glutamic Acid or Glycine
XXXXXS

SEQ ID NO: 36 - protein, 32 aa, Babesia microti
  VARIANT (6)...(6): Xaa = Methionine or Isoleucine
RCLSIXRFXXSXXTFIXIXXXMXFFXXXXXFL

SEQ ID NO: 37 - DNA, 1820 bp, Babesia microti
cggcacgagtagcccccaccatcttttgcattcatttcaagtttctccaaatctcgatgggacctccaattttggctccaccacaaacaagtctgacatattgagcaaaacatattgatttaatttaaagaacagacatctggccattcatgctaagaggtctcttcattgttgagtgggaacagccttgtatacgggcttacaacacaatggaaaaacaccttgtagaagagatcatgcttcactcagtgctagatgttgatgccagtgatttgcttggggtagtaagccagtactagaatacaggatgcacttggactggcaaacagaatacacctgttgcctgaatagaaactcacagagacccgatgctgtctggtaccaacaaggttctgcttctgggaagaatttacagatattatgttgggaaaagagacaccctgtatgtgtagaaacaaagaagcacagatcttagatgaattaatataagaatgatacttctctagaaacaaatgtagttaccaactatattccagaacccaatgcggattcagaatctgtacatgttgaaatccaggaacatgataacatcaatccacaagacgcttgcgatagtgagccgctcgaacaaatggattctgataccagggtgttgcccgaaagtttggatgagggggtaccacaccaattctctagattagggcaccactcagacatggcatctgatataaatgatgaagaaccatcatttaaaatcggcgagaatgacataattcaaccaccctgggaagatacagctccataccattcaatagatgatgaagagcttgacaacttaatgagactaacggcgcaagaaacaagtgacgatcatgaagaagggaatggcaaactcaatacgaataaaagtgagaagactgaaagaaaatcgcatgatactcagacaccgcaagaaatatatgaagagcttgacaacttactgagactaacggcacaagaaatatatgaagagcgtaaagaagggcatggcaaacccaatacgaataaaagtgagaaggctgaaagaaaatcgcatgatactcagacaacgcaagaaatatgtgaagagtgtgaagaagggcatgacaaaatcaataagaataaaagtggaaatgctggaataaaatcgtatgatactcagacaacgcaagaaatatgtgaagagtgtgaagaagggcatgacaaaatcaataagaataaaagtggaaatgctggaataaaatcgtatgatactcagacaccgcaggaaacaagtgacgctcatgaagaagggcatgacaaaatcaatacgaataaaagtgagaaggctgaaagaaaatcgcatgatactcagacaacgcaagaaatatgtgaagagtgtgaagaagggcatgacaaaatcaataagaataaaagtggaaatgctggaataaaatcgtatgatactcagacaccgcaggaaacaagtgacgctcatgaagaagagcatggcaatctcaataagaataaaagtgggaaggctggaataaaatcgcataatactcagacaccgctgaaaaaaaaagacttttgtaaagaagggtgtcatggttgcaataataagcccgaggataatgaaagagacccgtcgtcgcctgatgatgatggtggctgcgaatgcggcatgacgaatcactttgtctttgactacaagacaacactcttgttaaagagcctcaagactgaaacatccactcattattacattgccatggctgcaatttttactatttcattattcccatgcatgtttaaggctttcc

SEQ ID NO: 38 - protein, 445 aa, Babesia microti
YKNDTSLETNVVTNYIPEPNADSESVHVEIQEHDNINPQDACDSEPLEQMDSDTRVLPESLDEGVPHQFSRLGHHSDMASDINDEEPSFKIGENDIIQPPWEDTAPYHSIDDEELDNLMRLTAQETSDDHEEGNGKLNTNKSEKTERKSHDTQTPQEIYEELDNLLRLTAQEIYEERKEGHGKPNTNKSEKAERKSHDTQTTQEICEECEEGHDKINKNKSGNAGIKSYDTQTTQEICEECEEGHDKINKNKSGNAGIKSYDTQTPQETSDAHEEGHDKINTNKSEKAERKSHDTQTTQEICEECEEGHDKINKNKSGNAGIKSYDTQTPQETSDAHEEEHGNLNKNKSGKAGIKSHNTQTPLKKKDFCKEGCHGCNNKPEDNERDPSSPDDDGGCECGMTNHFVFDYKTTLLLKSLKTETSTHYYIAMAAIFTISLFPCMFKAF

SEQ ID NO: 39 - protein, 32 aa, Babesia microti
  VARIANT (3)...(3): Xaa = Glycine or Aspartic Acid
GHXKXNXNKSXXAXXKSXDTQTXQEXXXXXEE

SEQ ID NO: 40 - DNA, 2430 bp, Babesia microti
tgtattgtgtagataaaaatgatgtttcattatggaaatcaaaacctataacaactgtcagtaccactaatgatactattacaaatacacacactactaatgtaattaatgccaatcttattggccactttaattataaggatagggaacctttaacaatagtatttgtatacatgatcgatgaatcagaacaaaataaattatcacatccgaataaaattgataaaatcaaaatttctgattatataattgaatttgatgacaatgctaaattaccaactggtagtgttattgatttaaacatctatacttgcaaacataataatccagtattaattgaattttatgtttctatagaaggatctttctgctattatttctctcattgaataatgatacaaatgaatggaataatcacaaaataaaatatgataaaaaatataaagaatatacggacatgaatggtattcattattattatattgatggtagtttacttgtaagtggcgaagttacatctaattttcgttatatttctaaagaatatgaatatgagcatacaggattagtaaaaaaatattgtaatgaagaaagatgtgtaaaattggataacattaagataaaggataataatttggaaatttatgtgaaataatttaatgaagtataatattatttataataattcaaagattaatataatcaattattataattacaaaaataattaattgtagaatattatattattaatcaattcagattataaatacatatttttacatacatttcaatttaaacattcaaattaatgtcatttttatctacattattataattataactataatattcattaaatactattaaaaaaaatatcctctacattatattaattattatagtatgtcattatataacatattcacaacgtataacaaatcaatcattaacatatacatatatgatatcattaataatcaatatttaattgatacaataatcaatagtcatctgtaatataatcattgtatactaatttattataaattattacaaaatacactcttttacttcattttatttctgttaaatttcatattctaatattatattcatctttctcatgttactttaatctatttccatatttatcccaatttcttcatttaagactgagatgttcgttcgttcatacataaataatgtgtaaattttgtaatatataataatgtatacatctggtattacatctattttgtaataaatattaaaaaaacggttaaagttagtgccttaattccaggaattattacattagaaactttggtgattttagtgatttcggtgatcattgaaagaaatggtttgaaacttgcaatactgtcatactcatcataatccccaatgttggaaatcatgatgtcaacaattttattaaattcttctgctgcactattcaactccttaatcatgtcctcaaaatgagtgttataatctccatcctttttagtgatcttatccctcaaaactaaagctttagatttggattcgtcaaaatttttcttgatatcattaacggtattgtcataatagaatttatagattaaatgttgtaataataagtcacaatatataaacatatctttaagtacaatagacttccatatattacggaaatggtcaaaattatcagcagctggaccttccaatgtaccataggccttgtttgatatttcatcaaccaataacttatattttgaagagatagtggatgcattatcaaatattctagccaattcttctttcttcataagggaatattgttcaggaaaacatttttccaattcttttttcaatttattcttctccttggttttttcttcaatgtagtctttatgaccatcgttcaccctatctcgttccaatatcataacactatgtttgtatatataagataaacaaacttcattaaatataactattcttctagaatacggaagaagctgatatccaaatcgttcactagaccaaccagcttcactaggccaaccagttccactaggccaaccagttccactaggcccaccagcttcactaggcccaccagcttcactaggcccaccagcttcactaggcccaccagcttcactaggccaaccagttccactaggcccaccagcttcactaggcccaccagcttcactgggcccaacagttccactaggcccaccagcttcactaggcccaccagcttcgggatcggtatcacttgcaaagacagcaccgctcattaaaaagagtgtaatataaggaactaatattgatttaaatgacaccatctttataaaccatagttattggtacattattagtacattattggtatatgattggtacgtggtagtgattgtggtgctgcatctagtt

SEQ ID NO: 41 - protein, 128 aa, Babesia microti
YCVDKNDVSLWKSKPITTVSTTNDTITNTHTTNVINANLIGHFNYKDREPLTIVFVYMIDESEQNKLSHPNKIDKIKISDYIIEFDDNAKLPTGSVIDLNIYTCKHNNPVLIEFYVSIEGSFCYYFSH

SEQ ID NO: 42 - DNA, 1271 bp, Babesia microti
tgagaaaacgcatataattgtaactacgccagagaagtttgacgtagttacacgtaaaacaggcaatgagcccctgcttgagcggcttagattggttataattgatgaaatacacctactccatgacactaggggtccagtgctggaggctattgtggcccgcctgagtcagaggcccgaacgcgtaaggctagttggtctatcggccacgcttccaaactacgaagacgtggctagatttctcactgttaatctagaccgagggcttttctactttggcagccactttaggcctgtgcccttggagcaggtgtattatggcgtgaaggagaagaaggctatcaaacgtttcaacgcaatcaacgaaattctctaccaagaggtgattaacgatgtttctagctgccaaattcttgtttttgtgcattctagaaaggaaacgtacaggacggcaaaatttatcaaagacacggccctttcacgggacaacttgggagcctaaaccctaaaccctaaaccctaaaccctaaccctaaaccctaaaccctaaaccctaaaccctaaaccctaaccctaaccctaaccctaaccctaacctagccttcattgacgtctatccccaatcttagaaaaatcttcaaatcgattctagaataactggaagcaattatcagaaattgtataactgcttattagcttattagcttattagttaggatgtatgcacattgatgacaactagatgcagcaccacaatcactaccacgtaccaatcatataccaataatgtactaataatgtaccaataactatggtttataaagatggtgtcatttaaatcaatattagttccttatattacactctttttaatgagcggtgctgtctttgcaggtgataccgatcgcgaagctggtgggcctagtggaactgttgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtggaactggttggcctagtgaagctggtgggcctagtgaagctggtgggcctagtgaagctggtgggcctagtggaactggttggcctagtgaagctggttggcctagtgaagctggttggcctagtgaagctggttggcctagtgaagctggttggcctagtgaagctggttggcctagtgaa

SEQ ID NO: 43 - protein, 166 aa, Babesia microti
EKTHIIVTTPEKFDVVTRKTGNEPLLERLRLVIIDEIHLLHDTRGPVLEAIVARLSQRPERVRLVGLSATLPNYEDVARFLTVNLDRGLFYFGSHFRPVPLEQVYYGVKEKKAIKRFNAINEILYQEVINDVSSCQILVFVHSRKETYRTAKFIKDTALSRDNLGA

SEQ ID NO: 44 - protein, 154 aa, Babesia microti
LWFIKMVSFKSILVPYITLFLMSGAVFAGDTDREAGGPSGTVGPSEAGGPSEAGGPSEAGGPSEAGGPSEAGGPSEAGGPSEAGGPSEAGGPSGTGWPSEAGGPSEAGGPSEAGGPSGTGWPSEAGWPSEAGWPSEAGWPSEAGWPSEAGWPSE

SEQ ID NO: 45 - DNA, 4223 bp, Babesia microti
ctcgtgcctttctcaactgataacagctaacaaaaagtctcttatcttaaaccatcctatacctcgtattataatatgaaaagggccttttctaaatctttccccaaagttctgctatttaattaaaaaaaaaaaagactcattcaataaacgggtggggcagaaagggtacctttccaagtgttcttccatgacgacccacaatgcaaagttcttcttacaaagaaaagagaaagatccactgagtgataagtaacccagctggggccgggcggtggtggcgcacacctttaatcccagcactcgggaggcagaggcaggcggatctctgtgagttcgagaccaggctggaccgacagcctccaaaacaatacagagaaaccctgtctcataaaaaaccaaaaaaaaagtaacccagctggatttggtaactgtctcagaaacagactatataaaacctcatcaccctacaacaagtaggaagctagcgctccccaccccatcccaacacacacacacacacacacacacacacacacacacacacacacacgcacacacgcacgcacgcacacacgcacgcacgcacacacgcacacacgcacgcacacacgcacacacgcacgcacgcacgcacgcacgcacgcacgcacgcccttctgtgtctgttctgttcaagaagggtaccacaaaaaagtaccttatggccacatcaatgacaattattactgtatataaaatgcccccatggatggcattgtattgtcgaaattaaaggcacccccgaaagaacagcacagaggggctaccaccaattaactcccaggaggaaataaagacagaagtgtgaaggagggagagagggagggaggaagggagggagaaaaggagggaaaggaacaaggagtaacagggacaaaagcagcagatggtgccaggcaggagtgtgcctaccacaccgggccttcccgttacttcatttactctcctttgcagcctgggaataaacaagtcacgcgtcacccggtgtctcaagctcagcatggcttgatctgagtgcccgtgtatgtgttcattctataactgatttaaggaacaactttctgctcattgcctctatcttctcaaacatttcgaagcagttattttttataagaaaatataaaacaggccgactaaattcgatctttctctccccagctgctagtttcttatctagctgctttaggcagtctccacagattgcagccaggcccctattctcaattccatctgacttctgacagcgctctccatttcttatttgcagcttagacatcttcactgagagcaggagtaattcattcaaatgacaatgaggtatctgaatatcacacaaacacttcaaattctgtttattggaaatagatctgctcctgccccatcataacaatcctttttatcttacttaacaggggcaagaaaatctttcacttcatttcctatcatctcaaatgagttcctgtacatgaatgacttaaggtaaccatatccaacaacttgaagccaaccagtccctggtcctactacagacgttagggaacatatgtgaaaacctggtgtacaacctaaatcataactagacagaagacagcactatttcctggtcacatagaaagcagaatagcatcctcacaccaatgaggaaaatgtcatgaaggcaggagagatcatgactgaggtgatacttttaccaaagacttgccagtgattaatttctcaattagttagcaaaaaatatggctctctagtgaatttgtgtccacaccattttccagatgttttgatgtcacttaaatcaatctaattatttaagttaaaaaatgttacagatcattgctttttttcttttttagaagacatcaaaacaataggatttctatgaaatattctcacttcacagctgtgtcagttaaagtgctttgggttatacataaagaaaacagactcaagaaagtaagaacaggaatttggagcttgcaacactgatgttctttgtaaaaagagagactttatccagggattagattctgtcacaaggcctggaactctctcttctcagccttatttccccaatatggattagaatcttacactgcaagcttcccacaagggtggacaggtcctcaccatttgtttcagcaggaaaaagagtctgtatgcatccgtgatatctaagtcacaattccagaagtgagctttcctggctcctattggtcggacttaggtcaggtgtcacatttccttttggattagtctgtgattaatgaatgggcccactttgctcacccattaagacaataggcttccattctcgaagctggaagcatgacatgtcccacagaaactgtaataagagagaacataggttgctgtgtggagaaacgaggcaaccggcaagtcataagatgacaaagtcttggaaagtctaagtcagtggttctcagccttccctaaaccctaaaccctaaaccctaaaccctaaaccctaaaccctaaacccctaaaccctaaaccctaaaccctaaaccctaaaccctaaccctaaaccctaaaccctaaaccctaaaccctaaaccctaaccctaaccctaaccctaaccctaacctagccttcattgacgtctatccccaatcttagaaaaatcttcaaatcgattctagaataactggaagcaattatcagaaattgtataactgcttattagcttattagcttattagttaggatgtatgcacattgatgacaactagatgcagcaccacaatcactaccacgtaccaatcatataccaataatgtactaataatgtaccaataactatggtttataaagatggtgtcatttaaatcaatattagttccttatattacactctttttaatgagcggtgctgtctttgcaggtgataccgatcgcgaagctggtgggcctagtggaactgttgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtggaactgttgggcctagtgaagctggtgggcctagtgaagctggtgggcctagtgaagctggtgggcctagtgaagctggttggcctagtgaagctggttggcctagtgaagctggttggcctagtgaagctggttggcctagtgaagctggttggcctagtgaagctggttggcctagtgaacgatttggatatcagcttctttggtattctagaagaatagttatatttaatgaaatttatttatctcatatatacgaacatagtgttatgatattggaacgagatagggtgaacgatggtcataaagactacattgaagaaaaaaccaaggagaagaataaattgaaaaaagaattggaaaaatgttttcctgaacaatattcccttatgaagaaagaagaattggctagaataattgataatgcatccactatctcttcaaaatataagttattggttgatgaaatatccaacaaagcctatggtacattggaaggtccagctgctgatgattttgaccatttccgtaatatatggaagtctattgtacctaaaaatatgtttctatattgtgacttattattaaaacatttaatccgtttaacccccagaaagagctgaccagacaaaggttaactcttgaatcccaggcatcagcctgggaatccatcatgggactgatcaagaccccctgaatgtgggtgtcagtgaggaggcctaggtaatctattgagcctcgggcagcagatcagtacccatcccaattatacacaattgcagtgttgtggtttcacagtgaataattgtaggtcacagtccattatattgatgtcacagtttttaattgtcatgtcacagtgcaagctagtgatgtcagagtgtataactgtgttcatagagaatgtattgatgtcacagtcaataatcgtgatgtcatagtgcagtatattgatgtcacaatgtataattgtgatgttaaagtgcaagatagtgaagtcacagtatataattgtgatgtcatattgcattataatgatgtcacactttataattttttacatacagcactatagtgatgtaacagccaataattgtgatg

SEQ ID NO: 46 - protein, 294 aa, Babesia microti
LWFIKMVSFKSILVPYITLFLMSGAVFAGDTDREAGGPSGTVGPSEAGGPSEAGGPSEAGGPSEAGGPSEAGGPSEAGGPSEAGGPSGTVGPSEAGGPSEAGGPSEAGGPSEAGWPSEAGWPSEAGWPSEAGWPSEAGWPSEAGWPSERFGYQLLWYSRRIVIFNEIYLSHIYEHSVMILERDRVNDGHKDYIEEKTKEKNKLKKELEKCFPEQYSLMKKEELARIIDNASTISSKYKLLVDEISNKAYGTLEGPAADDFDHFRNIWKSIVPKNNFLYCDLLLKHLIRLTPRKS

SEQ ID NO: 47 - protein, 30 aa, Artificial Sequence
  Synthetic peptide of repeat region of antigen BMNI-3 (SEQ ID NO3)
SEAGGPSEAGGPSGTGWTSGTGWPSEAGWS

SEQ ID NO: 48 - protein, 30 aa, Artificial Sequence
  Synthetic peptide of repeat region of antigen BMNI-3 (SEQ ID NO3)
EAGGPSGTVGPSGTGWPSEAGWGSEAGWSS

SEQ ID NO: 49 - protein, 367 aa, Babesia microti
MVSFKSILVPYITLFLMSGAVFASDTDPEAGGPSEAGGPSGTVGPSEAGGPSEAGGPSGTGWPSEAGGPSEAGGPSEAGGPSEAGGPSGTGSEAGGWPSGTGWPSEAGWSSERFGYQLLPYSRRIVIFNEVCLSYIYKHSVMILERDRVNDGHKDYIEEKTKEKNKLKKELEKCFPEQYSLMKKEELARIFDNASTISSKYKLLVDEISNKAYGTLEGPAADNFDHFRNIWKSIVLKDMFIYCDLLLQHLIYKFYYDNTVNDIKKNFDESKSKALVLRDKITKKDGDYNTHFEDMIKELNSAAEEFNKIVDIMISNIGDYDEYDSIASFKPFLSMITEITKITKVSNVIIPGIKALTLTVFLIFITK

SEQ ID NO: 50 - DNA, 1908 bp, Babesia microti
aaaagatttaatgaacatactgacatgaatggtattcattattattatattgatggtagtttacttgcgagtggcgaagttacatctaattttcgttatatttctaaagaatatgaatatgagcatacagaattagcaaaagagcattgcaagaaagaaaaatgtgtaaatgtggataacattgaggataataatttgaaaatatatgcgaaacagtttaaatctgtagttactactccagctgatgtagcgggtgtgtcagatggattttttatacgtggccaaaatcttggtgctgtgggcagtgtaaatgaacaacctaatactgttggtatgagtttagaacaattcatcaagaacgagctttattcttttagtaatgaaatttatcatacaatatctagtcaaatcagtaattctttcttaataatgatgtctgatgcaattgttaaacatgataactatattttaaaaaaagaaggtgaaggctgtgaacaaatctacaattatgaggaatttatagaaaagttgaggggtgctagaagtgaggggaataatatgtttcaggaagctctgataaggtttaggaatgctagtagtgaagaaatggttaatgctgcaagttatctatccgccgcccttttcagatataaggaatttgatgatgaattattcaaaaaggccaacgataattttggacgcgatgatggatatgattttgattatataaatacaaagaaagagttagttatacttgccagtgtgttggatggtttggatttaataatggaacgtttgatcgaaaatttcagtgatgtcaataatacagatgatattaagaaggcatttgacgaatgcaaatctaatgctattatattgaagaaaaagatacttgacaatgatgaagattataagattaattttagggaaatggtgaatgaagtaacatgtgcaaacacaaaatttgaagccctaaatgatttgataatttccgactgtgagaaaaaaggtattaagataaacagagatgtgatttcaagctacaaattgcttctttccacaatcacctatattgttggagctggagttgaagctgtaactgttagtgtgtctgctacatctaatggaactgaatctggtggagctggtagtggaactggaactagtgtgtctgctacatctactttaactggtaatggtggaactgaatctggtggaacagctggaactactacgtctagtggaactgaagctggtggaactagtggaactactacgtctagtggagctgctagtggtaaagctggaactggaacagctggaactactacgtctagtgaaggtgctggtagtgataaagctggaactggaactagtggaactactacgtctagtggaactggtgctggtggagctggtagtggtggacctagtggacatgcttctaatgcaaaaattcctggaataatgacactaactctatttgcattattaacatttattgtaaattgaatgaaacacatgatttatacattattatatattacaaaatttacacattatttatgtatgaacgaacgaacatcttgctcttaaataaagaaattgagatatatatggaaatagattaaagtaacatgagaaagatgaatataatattagaatatgaaatttaacagaaataaaatgaagtaaaagagtgtattttgtaataatttataataaattagtatacaatgattatattacaaatggctattaaatattttattaattaaatattgattagtaatgatattatgtatgtacatgttagggttgattgttatacattgtgaatatattatataattgtatattatattgattgatataatgtagaggatatttttttaaatagtatttaat

SEQ ID NO: 51 - DNA, 1460 bp, Babesia microti
aatccaacatctagcctagttagtatatataggttaatatcacattatagattatctttggatgattggttattatataacatgtcgctgaatgacgattattttgctagataatataactaccggtgattctgaggacctactttaaagagaataattaacatatctaccagaatcagttccaatttatgtattttaaagctaatcactactcgaaaactacggtgaaaatggaaaaacaagtggaagctgtatgtcgtggaaagtcactacattttatgtgggcaaatttaataattctaaatactatgtttttgatgttaaaaagcgaaaaacacactttaatgcacattttaacatcatctgtataatatatatatcagcgttgaaatcatatggcaaaggtaataaagcgttacattttgagcgaataaaggcacatatgcaaacgtatgaagccttgtatatttgtggaattatattatgctagtaatttgtgattaataatggcaatatttatatacaaatattcgagcgttctattatatgcatgcacataattaatcacaaactctcatatcatggggcggtttcgcccatcataaacattactgttagcactctggtagattagcatggtgaatctctcgatacctgggctactgttgctttccgcatattccttaaattctgcaagtgcgggggatgtatatgagatatcttctggtaatccacccgacatagagccaacatctacttctctagaaacaaatgtagttaccaactatattccagaacccaatgcggattcagaatctgtacatgttgaaatccaggaacatgataacatcaatccacaagacgcttgcgatagtgagccgctcgaacaaatggattctgataccagggtgttgcccgaaagtttggatgagggggtaccacaccaattctctagattagggcaccactcagacatggcatctgatataaatgatgaagaaccatcatttaaaatcggcgagaatgacataattcaaccaccctgggaagatacagctccataccattcaatagatgatgaagagcttgacaacttaatgagactaacggcgcaagaaacaagtgacgatcatgaagaagggaatggcaaactcaatacgaataaaagtgagaagactgaaagaaaatcgcatgatactcagacaccgcaagaaatatatgaagagcttgacaacttactgagactaacggcacaagaaatatatgaagagcgtaaagaagggcatggcaaacccaatacgaataaaagtgagaaggctgaaagaaaatcgcatgatactcagacaacgcaagaaatatgtgaagagtgtgaagaagggcatgacaaaatcaataagaataaaagtggaaatgctggaataaaatcgtatgatactcagacaccgcaggaaacaagtgac

SEQ ID NO: 52 - protein, 503 aa, Babesia microti
KRFNEHTDMNGIHYYYIDGSLLASGEVTSNFRYISKEYEYEHTELAKEHCKKEKCVNVDNIEDNNLKIYAKQFKSVVTTPADVAGVSDGFFIRGQNLGAVGSVNEQPNTVGMSLEQFIKNELYSFSNEIYHTISSQISNSFLIMMSDAIVKHDNYILKKEGEGCEQIYNYEEFIEKLRGARSEGNNMFQEALIRFRNASSEEMVNAASYLSAALFRYKEFDDELFKKANDNFGRDDGYDFDYINTKKELVILASVLDGLDLIMERLIENFSDVNNTDDIKKAFDECKSNAIILKKKILDNDEDYKINFREMVNEVTCANTKFEALNDLIISDCEKKGIKINRDVISSYKLLLSTITYIVGAGVEAVTVSVSATSNGTESGGAGSGTGTSVSATSTLTGNGGTESGGTAGTTTSSGTEAGGTSGTTTSSGAASGKAGTGTAGTTTSSEGAGSDKAGTGTSGTTTSSGTGAGGAGSGGPSGHASNAKIPGIMTLTLFALLTFIVN

SEQ ID NO: 53 - protein, 275 aa, Babesia microti
MVNLSIPGLLLLSAYSLNSASAGDVYEISSGNPPDIEPTSTSLETNVVTNYIPEPNADSESVHVEIQEHDNINPQDACDSEPLEQMDSDTRVLPESLDEGVPHQFSRLGHHSDMASDINDEEPSFKIGENDIIQPRWEDTAPYHSIDDEELDNLMRLTAQETSDDHEEGNGKLNTNKSEKTERKSHDTQTPQEIYEELDNLLRLTAQEIYEERKEGHGKPNTNKSEKAERKSHDTQTTQEICEECEEGHDKINKNKSGNAGIKSYDTQTPQETSD

SEQ ID NO: 54 - DNA, 22 bp, Artificial Sequence
  PCR Primer
tttgcaggtgataccgatcgcg

SEQ ID NO: 55 - DNA, 24 bp, Artificial Sequence
  PCR Primer
tggtattctagaagaatagttata

SEQ ID NO: 56 - DNA, 306 bp, Babesia microti
ttgcaggtgataccgatcgcgaagctggtgggcctagtggaactgttgggcccagtgaagctggtgggcctagtgaagctggtgggcctagtggaactgttgggcccagtgaagctggtgggcctagtgaagctggtgggcctagtggaactggttggcctagtgaagctggtgggcctagtggaactgttgggcccagtgaagctggtgggcctagtgaagctggtgggcctagtggaactggttggcctagtggaactggttggcctagtgaagttggttggcccattgaaccatttggatatc

SEQ ID NO: 57 - DNA, 318 bp, Babesia microti
ttgcaggtgataccgatcgcgaagctggtgggcctagtggaactgttgggcccagtgaagctggtgggcctagtgaagctggtgggcctagtggaactgttgggcccagtgaagctggtgggcctagtgaagctggtgggcctagtggaactggttggcctagtgaagctggtgggcctagtggaactgttgggcccagtgaagctggtgggcctagtgaagctggtgggcctagtggaactggttggcctagtggaactggttggcctagtgaagttggttggcctaatgaaccatttggatatcaccttctttggt

SEQ ID NO: 58 - DNA, 358 bp, Babesia microti
ttgcaggtgataccgatcgcgaagctggtgggcctagtggaactgttgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtgaagctggttggcctagtgaagctggttggcctagtgaagctggtgggcctagtggaactggttggcctagtgaagctggttggcctagtgaagctggttggcctagtgaagctggttggcctagtgaagctggttggcc

SEQ ID NO: 59 - DNA, 409 bp, Babesia microti
tgcaggtgataccgatcgcgaagctggtgggcctagtggaactgttgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtgaagctggttggcctagtgaagctggttggcctagtgaagctggtgggcctagtggaactggttggcctagtgaagctggttggcctagtgaagctggttggcctagtgaagctggttggcctagtgaagctggttggcctagtgaacgatttggatatcagcttctttggtattctagaagaatagttata

SEQ ID NO: 60 - DNA, 351 bp, Babesia microti
gtgaagctggtgggcctagtggaactgttgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtgaagctggttggcctagtgaagctggttggcctagtgaagctggtgggcctagtggaactggttggcctagtgaagctggttggcctagtgaagctggttggcctagtgaagctggttggcctagtgaagctggttggcctagtgaacgat

SEQ ID NO: 61 - DNA, 410 bp, Babesia microti
aggtgataccgatcgcgaagctggtgggcctagtggaactgttgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtgaagctggttggcctagtgaagctggttggcctagtgaagctggtgggcctagtggaactggttggcctagtgaagctggttggcctagtgaagctggttggcctagtgaagctggttggcctagtgaagctggttggcctagtgaacgatttggatatcagcttctttggtattcta

SEQ ID NO: 62 - DNA, 416 bp, Babesia microti
ttgcaggtgataccgatcgcgaagctggtgggcctagtggaactgttgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtgaagctggttggcctagtgaagctggttggcctagtgaagctggtgggcctagtggaactggttggcctagtgaagctggttggcctagtgaagctggttggcctagtgaagctggttggcctagtgaagctggttggcctagtgaacgatttggatatcag

SEQ ID NO: 63 - DNA, 356 bp, Babesia microti
ttgcaggtgataccgatcgcgaagctggtgggcctagtggaactgttgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtggaactggttggcctagtgaagctggttggcctagtgaagctggttggcctagtgaagctggttggcctagtgaagctggttggcctagtgaagctggttggcctagtgaacgatttggatatcagcttctttggtattctagaagaatagttata

SEQ ID NO: 64 - DNA, 285 bp, Babesia microti
ttgcaggtgataccgatcgcgaagctggtgggcctagtggaactgttgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtggaactggttggcctagtgaagctggttggcctagtgaagctggttggcctagtgaagctggttggcctagtgaagctggttggcctagtgaagctggttggc

SEQ ID NO: 65 - DNA, 342 bp, Babesia microti
ttgcaggtgataccgatcgcgaagctggtgggcctagtggaactgttgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtggaactggttggcctagtgaagctggttggcctagtgaagctggttggcctagtgaagctggttggcctagtgaagctggttggcctagtgaagctggttggcctagtgaacgatttggatatcagcttctttggtattcta

SEQ ID NO: 66 - DNA, 363 bp, Babesia microti
ttgcaggtgataccgatcgcgaagctggtgggcctagtggaactgttgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtggaactggttggcctagtgaagctggttggcctagtgaagctggttggcctagtgaagctggttggcctagtgaagctggttggcctagtgaagctggttggcctagtgaacgatttggatatcagcttctttggtattctagaa

SEQ ID NO: 67 - DNA, 363 bp, Babesia microti; identical to SEQ ID NO: 66

SEQ ID NO: 68 - protein, 101 aa, Babesia microti
AGDTDREAGGPSGTVGPSEAGGPSEAGGPSGTVGPSEAGGPSEAGGPSGTGWPSEAGGPSGTVGPSEAGGPSEAGGPSGTGWPSGTGWPSEVGWPIEPFGY

SEQ ID NO: 69 - protein, 105 aa, Babesia microti
AGDTDREAGGPSGTVGPSEAGGPSEAGGPSGTVGPSEAGGPSEAGGPSGTGWPSEAGGPSGTVGPSEAGGPSEAGGPSGTGWPSGTGWPSEVGWPNEPFGYHLLW

SEQ ID NO: 70 - protein, 118 aa, Babesia microti
AGDTDREAGGPSGTVGPSEAGGPSEAGGPSEAGGPSEAGGPSEAGGPSEAGGPSEAGGPSEAGGPSEAGGPSEAGWPSEAGWPSEAGGPSGTGWPSEAGWPSEAGWPSEAGWPSEAGW

SEQ ID NO: 71 - protein, 136 aa, Babesia microti
AGDTDREAGGPSGTVGPSEAGGPSEAGGPSEAGGPSEAGGPSEAGGPSEAGGPSEAGGPSEAGGPSEAGGPSEAGWPSEAGWPSEAGGPSGTGWPSEAGWPSEAGWPSEAGWPSEAGWPSERFGYQLLWYSRRIVI

SEQ ID NO: 72 - protein, 116 aa, Babesia microti
EAGGPSGTVGPSEAGGPSEAGGPSEAGGPSEAGGPSEAGGPSEAGGPSEAGGPSEAGGPSEAGGPSEAGWPSEAGWPSEAGGPSGTGWPSEAGWPSEAGWPSEAGWPSEAGWPSER

SEQ ID NO: 73 - protein, 136 aa, Babesia microti
GDTDREAGGPSGTVGPSEAGGPSEAGGPSEAGGPSEAGGPSEAGGPSEAGGPSEAGGPSEAGGPSEAGGPSEAGGPSEAGWPSEAGWPSEAGGPSGTGWPSEAGWPSEAGWPSEAGWPSEAGWPSERFGYQLLWYS

SEQ ID NO: 74 - protein, 138 aa, Babesia microti
AGDTDREAGGPSGTVGPSEAGGPSEAGGPSEAGGPSEAGGPSEAGGPSEAGGPSEAGGPSEAGGPSEAGGPSEAGGPSEAGGPSEAGWPSEAGWPSEAGGPSGTGWPSEAGWPSEAGWPSEAGWPSEAGWPSERFGYQ

SEQ ID NO: 75 - protein, 118 aa, Babesia microti
AGDTDREAGGPSGTVGPSEAGGPSEAGGPSEAGGPSEAGGPSEAGGPSEAGGPSEAGGPSEAGGPSGTGWPSEAGWPSEAGWPSEAGWPSEAGWPSEAGWPSERFGYQLLWYSRRIVI

SEQ ID NO: 76 - protein, 94 aa, Babesia microti
AGDTDREAGGPSGTVGPSEAGGPSEAGGPSEAGGPSEAGGPSEAGGPSEAGGPSEAGGPSGTGWPSEAGWPSEAGWPSEAGWPSEAGWPSEAGW

SEQ ID NO: 77 - protein, 113 aa, Babesia microti
AGDTDREAGGPSGTVGPSEAGGPSEAGGPSEAGGPSEAGGPSEAGGPSEAGGPSEAGGPSEAGGPSGTGWPSEAGWPSEAGWPSEAGWPSEAGWPSEAGWPSERFGYQLLWYS

SEQ ID NO: 78 - protein, 120 aa, Babesia microti
AGDTDREAGGPSGTVGPSEAGGPSEAGGPSEAGGPSEAGGPSEAGGPSEAGGPSEAGGPSEAGGPSEAGGPSGTGWPSEAGWPSEAGWPSEAGWPSEAGWPSEAGWPSERFGYQLLWYSR

SEQ ID NO: 79 - protein, 120 aa, Babesia microti; identical to SEQ ID NO: 78

SEQ ID NO: 80 - DNA, 29 bp, Artificial Sequence
  PCR Primer
cagagcagtactgatgatattaagaaggc

SEQ ID NO: 81 - DNA, 43 bp, Artificial Sequence
  PCR Primer
caatatgaattcagtgaatatttacaataaatgttaataatgc

SEQ ID NO: 82 - DNA, 32 bp, Artificial Sequence
  PCR Primer
cataacaatattccagaacccaatgcggattc

SEQ ID NO: 83 - DNA, 32 bp, Artificial Sequence
  PCR Primer
cgctagaattcattagaaagccttaaacatgc